SEQUENCE LISTING (4 sequences)

SEQ ID NO: 1 - DNA, 33 bp, Artificial Sequence
  Primer
atggtcacaatgtcctccgttggttatggggat

SEQ ID NO: 2 - DNA, 33 bp, Artificial Sequence
  Primer
atccccataaccaacggaggacattgtgaccat

SEQ ID NO: 3 - DNA, 3534 bp, Homo sapiens
atggcaaatggtggcggcggcggcggcggcagcagcggcggcggcggcggcggcggaggcagcagtcttagaatgagtagcaatatccacgcgaaccatctcagcctagacgtgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctt

SEQ ID NO: 4 - protein, 1178 aa, Homo sapiens
MANGGGGGGGSSGGGGGGGGSSLRMSSNIHANHLSLDVSSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

(19) United States
(12) Patent Application Publication
(10) Pub. No.: US 2016/0184455 A1
(43) Pub. Date: Jun. 30, 2016

(54) COMPOSITIONS AND METHODS FOR TREATING SMOOTH MUSCLE DYSFUNCTION

(71) Applicants: ION CHANNEL INNOVATIONS, LLC, New York, NY (US); ALBERT EINSTEIN COLLEGE OF MEDICINE, INC., Bronx, NY (US)

(72) Inventors: Arnold Melman, New York, NY (US); Kelvin Davies, Bronx, NY (US); David Spray, Bronx, NY (US); Yi Wang, Bronx, NY (US); Joel Friedman, Bronx, NY (US); Mahantash Nadavi, Bronx, NY (US)

(21) Appl. No.: 14/910,176

(22) PCT Filed: Aug. 5, 2014
(86) PCT No.: PCT/US2014/049811

§ 371 Date: Feb. 4, 2016

Related U.S. Application Data
(60) Provisional application No. 61/862,306, filed on Aug. 5, 2013.

(51) Int. Cl. A61K 48/00; C07K 14/705

(52) U.S. Cl. CPC A61K 48/005; C07K 14/705

(57) ABSTRACT

The present disclosure provides compositions and methods to improve one or more signs or symptoms of smooth muscle diseases. Compositions of the disclosure may include a plasmid vector containing a variant nucleic that encodes for a variant amino acid sequence of the alpha subunit of the BK potassium channel. Compositions may further include a nanoparticle delivery system. Compositions and methods of use of the disclosure may be used to treat, for example, over active bladder (OAB) syndrome and erectile dysfunction (ED).

### RELATED APPLICATIONS

This application claims priority to and benefit of provisional application U.S. Ser. No. 61/862,306 filed on Aug. 5, 2013, the contents of which are herein incorporated by reference in its entirety.

### FIELD OF THE INVENTION

The present disclosure relates generally to the fields of molecular biology and electrophysiology as well as pharmaceutical and medical therapies to improve one or more signs or symptoms of smooth muscle dysfunction.

### BACKGROUND OF THE INVENTION

There are many physiological dysfunctions or disorders which are caused by the deregulation of smooth muscle tone. Included among these dysfunctions and disorders are: asthma; benign hyperplasia of the prostate gland (BHP); coronary artery disease (infused during angiography); erectile dysfunction; genitourinary dysfunctions of the bladder, endopelvic fascia, prostate gland, ureter, urethra, urinary tract, and vas deferens; irritable bowel syndrome; migraine headaches; premature labor; Raynaud's syndrome; and thromboangitis obliterans.

Erectile dysfunction is a common illness that is estimated to affect 10 to 30 million men in the United States. Among the primary disease-related causes of erectile dysfunction are aging, atherosclerosis, chronic renal disease, diabetes, hypertension and antihypertensive medication, pelvic surgery and radiation therapy, and psychological anxiety.

Abnormal bladder function is another common problem which significantly affects the quality of life of millions of men and women in the United States. Many common diseases (e.g., BHP, diabetes mellitus, multiple sclerosis, and stroke) alter normal bladder function. Significant untoward changes in bladder function are also a normal result of advancing age. There are two principal clinical manifestations of altered bladder physiology: the atonic bladder and the hyperreflexic bladder. The atonic bladder has diminished capacity to empty its urine contents because of ineffective contractility of the detrusor smooth muscle (the outer smooth muscle of the bladder wall). In the atonic state, diminished smooth muscle contractility is implicated in the etiology of bladder dysfunction. Thus, it is not surprising that pharmacological modulation of smooth muscle tone is insufficient to correct the underlying problem. In fact, the prevailing method for treating this condition uses clean intermittent catheterization; this is a successful means of preventing chronic urinary tract infection, pyelonephritis, and eventual renal failure. As such, treatment of the atonic bladder ameliorates the symptoms of disease, but does not correct the underlying cause.

Conversely, the hyperreflexic, or uninhibited, bladder contracts spontaneously; this may result in urge incontinence, where the individual is unable to control the passage of urine. The hyperreflexic bladder is a more difficult problem to treat. Medications that have been used to treat this condition are usually only partially effective, and have severe side effects that limit the patient's use and enthusiasm. The currently-accepted treatment options (e.g., oxybutynin and tolteradine) are largely nonspecific, and most frequently involve blockade of the muscarinic-receptor pathways and/or the calcium channels on the bladder myocytes. Given the central importance of these two pathways in the cellular functioning of many organ systems in the body, such therapeutic strategies are not only crude methods for modulating bladder smooth muscle tone; rather, because of their very mechanism(s) of action, they are also virtually guaranteed to have significant and undesirable systemic effects. Accordingly, there is a great need for improved treatment options for bladder dysfunction.

Despite multiple attempts to develop a cure or treatment for diseases caused by altered smooth muscle tone, current therapies are inadequate because they provide limited efficacy and/or significant side effects. Thus, there is a long-felt need in the art for a pharmaceutical and/or medical intervention to address the underlying cause of altered smooth muscle tone by increasing efficacy with minimal side effects.

### SUMMARY OF THE INVENTION

The compositions and methods of the disclosure employ gene transfer technology to restore normal smooth muscle function.

In one aspect the invention provides a mutated Maxi K channel nucleic acid having a single point mutation at nucleotide position 1054, when numbered in accordance with SEQ ID NO:3.

The invention provides a nucleic acid molecule including then nucleic acid sequence of SEQ ID NO:3 having a single point mutation at nucleotide position 1054. The point mutation results in serine at position 352 of SEQ ID No: 4. In some aspects, the nucleic acid molecule is operably-linked to a promoter. The promoter is not an urothelium specific expression promoter. For example, the promoter is a CMV promoter (VAX) or a smooth muscle specific expression promoter (SMAA).

Also provided by the invention are plasmids and vectors containing the nucleic acid molecules of the invention. The vector is for example, an adenovirus. The nucleic acid molecules, plasmids or vectors of the invention are associated with or conjugated to a nanoparticle. Further provided by the invention are delivery systems containing a plurality of nanoparticles or vectors according to the invention and a pharmaceutically acceptable diluent or carrier. The delivery system is suitable for topical administration to a subject. Alternatively, the delivery system is suitable for systemic administration to a subject.

Exemplary nucleic acid molecules include a pVAX-hSlo-T352S nucleic acid molecule; a pVAX-hSlo-T352S-C997 nucleic acid molecule; a pVAX-hSlo-T352S-C496A nucleic acid molecule; a pVAX-hSlo-T352S-C681A nucleic acid molecule; a pVAX-hSlo-T352S-M602L nucleic acid molecule; a pVAX-hSlo-T352S-M778L nucleic acid molecule; pVAX-hSlo-T352S-M805L nucleic acid molecule; pSMAA-hSlo-T352S nucleic acid molecule; a pSMAA-hSlo-T352S-C997 nucleic acid molecule; a pSMAA-hSlo-T352S-C496A nucleic acid molecule; a pSMAAhSlo-T352S-C681A nucleic acid molecule; a pSMAA-hSlo-T352S-M602L nucleic acid molecule; a pSMAA-hSlo-T352S-M778L nucleic acid molecule; and pSMAA-hSlo-T352S-M805L nucleic acid molecule.

Also provided are methods for expressing a variant BKα channel within a smooth muscle cell by contacting the cell with a nucleic acid molecule according to the invention.

The cell is contacted in vivo, ex vivo, or in vitro. The smooth muscle is for example a detrusor urinae muscle.

In further aspects the invention provides methods of treating smooth muscle dysfunction in a subject, by introducing into smooth muscle cells of the subject the nucleic acid molecule or the delivery system of according to the invention Thee nucleic acid is expressed in the smooth cells such that smooth muscle tone is regulated. The regulation of smooth muscle tone results in less heightened contractility of smooth muscle in the subject.

The subject has over active bladder (OAB) syndrome, erectile dysfunction (ED), asthma; benign hyperplasia of the prostate gland (BHP); coronary artery disease (infused during angiography); genitourinary dysfunctions of the bladder, endopelvic fascia, prostate gland, ureter, urethra, urinary tract, and vas deferens; irritable bowel syndrome; migraine headaches; premature labor; Raynaud's syndrome; and thromboangitis obliterans.

In a further aspect the invention provides methods of treating over active bladder (OAB) syndrome in a subject, by introducing into bladder smooth muscle cells of the subject the nucleic acid molecule or the delivery system of according to the invention. The nucleic acid is expressed in the bladder smooth cells such that bladder smooth muscle tone is regulated. The regulation of bladder smooth muscle tone results in less heightened contractility of smooth muscle in the subject.

In yet another aspect, the invention provides methods for treating penile flaccidity caused by heightened contractility of penile smooth muscle in a subject, byintroducing into penile smooth muscle cells of the subject he nucleic acid molecule or the delivery system of according to the invention. The nucleic acid expressed in the penile smooth muscle cells such that penile smooth muscle tone is regulated. Thhe regulation of penile smooth muscle tone results in less heightened contractility of penile smooth muscle in the subject.

Administration may be performed by, for example, injection or implantation. Routes of injection include, but are not limited to, subcutaneous, intravenous, intramuscular, or intrapelvic injections. Locations for implantation include, but are not limited to, subcutaneous, intravenous, intramuscular, or intrapelvic areas of the body. For example, a composition of the disclosure may be implanted within a pelvis, a bladder, and/or a penis of a subject. The nucleic acid molecule is introduced by naked DNA transfer. The delivery system is introduced by instillation into the lumen of the bladder.

Other features and advantages of the invention will be apparent from and are encompassed by the following detailed description and claims.

### BRIEF DESCRIPTION OF THE DRAWINGS

FIG. 1 is a Schematic depiction of the role of the MaxiK channel in modulating transmembrane calcium flux and free intracellular calcium concentration in a bladder smooth muscle cell.

FIG. 2 is a schematic diagram depicting the plasmid pVAX-hSLO. hSlo is under control of the CMV promoter positioned upstream of the transgene. The construct also contains the Bovine Growth Hormone poly A site, kanamycin resistance gene and pUC origin of replication. In another embodiment, hSlo may be placed under the control of a promoter that specifically expresses the gene in the smooth muscle of a targeted organ.

FIG. 3 is a graph depicting the effect of a point-mutation, T352S, in the pore of the hSlo channel on the channel's electrical properties. The T352S mutant hSlo channel displays significantly higher current compared to a wild type hSlo channel. 293 cells transfected with a sequence containing the T352S point mutation were used for this patch-clamp experiment. C1 represents T352S plus C(cytosine) 496A (alanine) mutant; C2 represents T352S plus C(cytosine)681A mutant; C3 represents T352S plus C(cytosine)977A mutant; M1 represents T352S plus M(methionine)602L (lysine) mutant; M2 represents T352S plus M(methionine)788L (lysine) mutant; M3 represents T352S plus M(methionine)805L (lysine) mutant.

FIG. 4 is a graph depicting the results of the patch clamp experiment described in Example 1. Each of the constructs depicted were transfected into HEK cells. The current was measured after 24-48 hours in a high glucose (22.5 mM) environment. The T352S single point mutation confers resistance to oxidative stress. The double point mutations (C1, C2, C3, M1, M2, and/or M3) may compromise the resistance of the T352S single point mutation to oxidative stress.

FIG. 5 is a chart showing the effect of different promoters on bladder function in the PUO model of OAB. [pVAX=vector only, pVAXuro-hSlo (hSlo expressed from the with uroplakin UPKII promoter), pVAX-hSlo (hSlo expressed from the CMV promoter), pSMAA-hSlo (hSlo expressed from the smooth muscle alpha actin promoter.) *=p<0.05]

FIG. 6. Representative cystometric, (A-D) organ bath (E-G) and patch clamp (H) studies from a control and a 2 month STZ-diabetic rat. Panel A and B: Cumulative volume of excreted urine. Panel C and D: Intravesical pressure. Panel E and F: Isometric recordings of bladder strip from control and diabetic bladder illustrating marked spontaneous phasic contractions in the diabetic strip, characteristic of detrusor overactivity. Panel G: Relative increase in amplitude of spontaneous contractions induced by treatment with increasing concentration of iberiotoxin (IBTX), a MaxiK channel blocker. Data represent an average from 5 animals. Panel H: Results from single-cell patch clamping studies with stepwise increases in voltage performed in detrusor SM cells isolated from control and 2 month STZ-rats with bladder hyperactivity before and after incubation of cells with 300 nM IBTX. Stepwise application of voltage across the cell membrane results in opening of channels and outward current flow. The mean ratio of the maximum current at a particular voltage (Imax) to Imax after incubation with 300 nM IBTX is shown.

FIG. 7. Spontaneous activity (SA) of PUO rat bladder. PUO rats were treated intravesically with empty pVAX (control) and pVAX for expression of wild type hSlo and mutant hSlo T352S genes. Our initial cystometry studies with PUO rats treated with 30 μg of pVAX-hSlo T352S indicate that when compared to our previously obtained data this hSlo mutant may be more efficient in reducing DO than the wild type gene (FIG. 4). Note the significantly higher effect of mutant hSlo T352S in reducing the bladder SA of PUO rats. Data correspond to mean±SEM; pVAX=14; pVAX-hSlo=17; pVAX-hSlo T352S=6; ANOVA followed by Dunnett's multiple comparison: *p<0.05, **p<0.01 vs control; Student's t-test, pVAX-hSlo vs pVAX-hSlo T352S, $ p<0.05)

FIG. 8 Panel A: Nanoparticles viewed by electron microscopy Panel B: FITC-labelled nanoparticles in solution, viewed by epifluo-rescence microscopy (20× magnification). Panel C. FITC-labeled nanoparticles were applied to the rat penis surface. One hour after application the animals were sacrificed and the penis cross-sectioned. Tissue sections were examined with an epifluorescence microscope at 4× and 20× (shown in inset) magnification. Fluorescent nanoparticles appear as small red spots and can be seen penetrating the penis periphery (dermis), as well as the cavernous vein lining and corpus spongiosum.

FIG. 9. In vivo, ex vivo and in vitro monitoring of gene expression. Panel A: Nanoparticles were generated encapsulating the mCherry plasmid, which expresses a red fluorescent protein, and were added to a culture of HeLa cells. After 7 hours the cells were visualized using phase contrast (left panel) and epifluorescence (middle panel) microscopy. Overlay of the two images (right panel) demonstrated that nearly all cells (approximately 95%) were expressing the mCherry fluorophore. Panel B: Nanoparticles were generated encapsulating the hMaxiK plasmid and added at different concentrations to a culture of HEK293 cells. After 20 hrs expression of hMaxiK gene was determined by qRT-PCR. Bars represent the average fold change in MaxiK expression over background from experiments repeated in triplicate. Panel C: Whole animal fluorescence imaging 3 days after saline injection (left) or pmCherry-N1 (right) into the detrusor. Panel D: Bladders from animals in panel C were removed and imaged for mCherry fluorescence. On the heat map the red color indicates higher fluorescence.

### DETAILED DESCRIPTION

The present invention is based upon the surprising discovery that a single point mutation in the alpha, or pore-forming, subunit of the human Maxi-K channel (hSlo) is more efficient in reducing detrusor overactivity (DO) in smooth muscle than the wild type hSlo gene. Specifically, a single point mutation at nucleotide position 1054 of the hSlo gene which results in a substitution of a Threonine (T) for a Serine (S) at position 352 of the amino acid sequence causes increased current of the MaxiK channel at lower intracellular calcium ion concentrations when compared to the channels expressed by the non-mutated gene. Unexpectedly, the single mutation had improved conductivity in high glucose of high oxidative stress environments compared to genes having multiple mutations.

Accordingly, the present invention provides compositions and methods of gene therapy for treating physiological dysfunctions of smooth muscle through the delivery into, and expression in, a smooth muscle cell of a mutated hSlo gene. As used herein, “regulation” is the modulation of relaxation or the modulation of contraction.

The MaxiK channel (also known as the BK channel) provides an efflux pathway for potassium ions from the cell, allowing relaxation of smooth muscle by inhibition of the voltage sensitive Ca²⁺ channel, and thereby effecting normalization of organ function by reducing pathological heightened smooth muscle tone. The terms “MaxiK channel” and “BK channel” are used interchangeably herein.

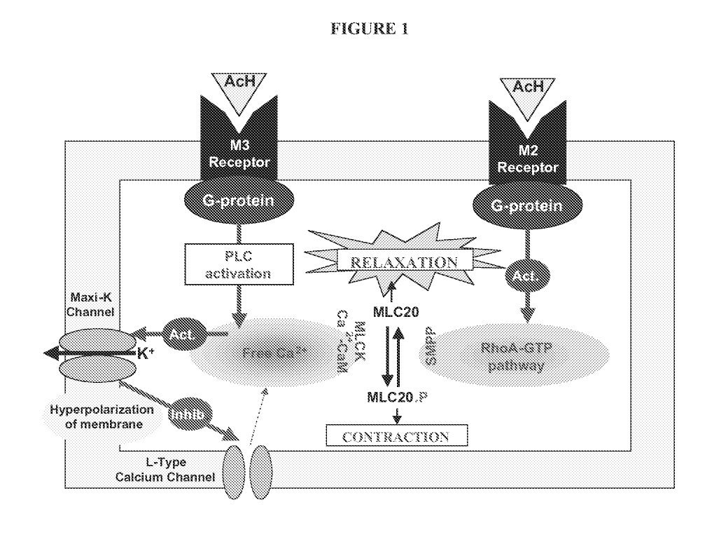

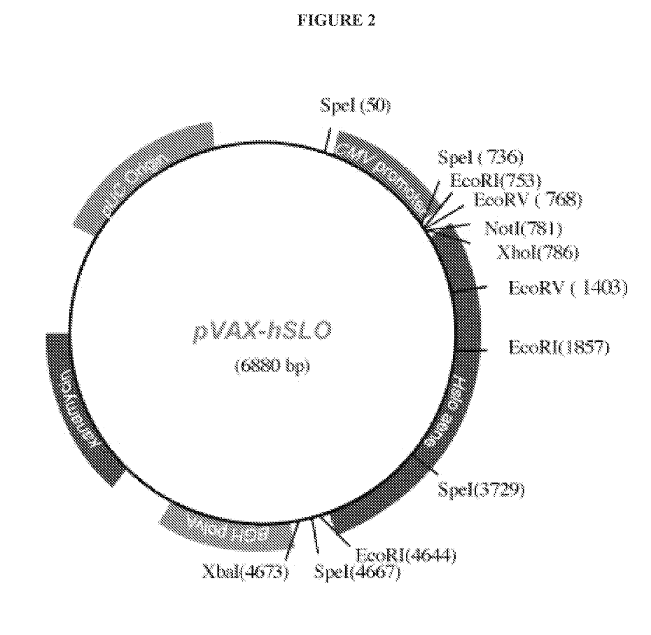

Strategic clusters of MaxiK channels in close proximity to the ryanodine-sensitive calcium stores of the underlying sarcoplasmic reticulum provide an important mechanism for the local modulation of calcium signals (i.e., sparks) and membrane potential in diverse smooth muscle, including urinary bladder (see FIG. 1). As shown in FIG. 1, the signal that activates a muscarinic M3 receptor causes an increase in intracellular calcium levels. The increase in the intracellular calcium level increases the open probability of the MaxiK channel, thus increasing the outward movement of K⁺ through the calcium sensitive MaxiK channel. The efflux of K⁺ causes a net movement of positive charge out of the cell, making the cell interior more negative with respect to the outside. This has two major effects. First, the increased membrane potential ensures that the calcium channel spends more time closed than open. Second, because the calcium channel is more likely to be closed, there is a decreased net flux of Ca²⁺ into the cell and a corresponding reduction in the free intracellular calcium levels. The reduced intracellular calcium promotes smooth muscle relaxation. The major implication of having more MaxiK channels in the cell membrane, therefore, is that it should lead to enhanced smooth muscle cell relaxation to any given stimulus for relaxation.

Increased intercellular communication among detrusor myocytes in occurs in both animal models of partial urethral obstruction (PUO) and humans with detrusor overactivity (DO). With respect to increased intercellular communication, the impact of increased calcium signaling may be augmented when compared to a normal bladder with potentially lower levels of intercellular coupling. This increased calcium signaling contributes, at least in part, to the “non-voiding contractions” observed in the PUO rat model. However, if there were a parallel increase in MaxiK channel expression (for example, as a result of over-expression of a MaxiK channel encoding transgene of a composition or method of the disclosure), then presumably the resultant recombinant and/or transgenic channels in expressed by these transfected cells may “short circuit” abnormally increased calcium signals, prevent further spread through gap junctions, and thus, prevent sufficient augmentation of abnormal and increased calcium signaling (by, for example, non-transfected myocyte recruitment) to produce detectable contractile responses. The induction of detectable contractile responses by over-expression of a MaxiK channel encoding transgene of a composition or method of the disclosure eliminates or ameliorates the non-voiding contractions characteristic of DO, the clinical correlate of urgency. Conversely, because the involvement of spinal reflexes in the micturition response produces coordinated detrusor contractions well in excess of the abnormally increased calcium signaling associated with DO, MaxiK transgene over-expression may effectively reduce or inhibit the weaker abnormally increased calcium signal that contributes to DO (as measured in an animal model as a decrease in IMP (intermicturition pressure) or SA (spontaneous activity) compared to control levels), without significantly or detectably affecting the more robust micturition contraction response.

Aging and disease can result in changes in the expression of the final product of the hSlo gene, the gene that expresses the α-subunit of the large conductance Ca2+-activated, voltage sensitive potassium (BKα) channel. Those changes result in reduced organ-specific physiological modification of the tone of the smooth muscle that comprises the organ. The effect is heightened tone of the organ that cause human disease such as erectile dysfunction (ED) in the penis, urinary urgency, frequency, nocturia, and incontinence in the bladder (e.g. over active bladder (OAB) syndrome), asthma in the lungs, irritable bowel in the colon, glaucoma in the eyes and bladder outlet obstruction in the prostate.

Modification of hSlo

Modifications of the hSlo gene may be used to effectively treat human disease that is caused, for example, by alterations of the BK channel expression, activity, upstream signaling events, and/or downstream signaling events. Modifications to a wild type nucleotide or peptide sequence of hSlo may include, but are not limited to, deletions, insertions, frameshifts, substitutions, and inversions. For example, contemplated modifications to the wild type sequence of hSlo include substitutions of a single nucleotide in a DNA, cDNA, or RNA sequence encoding hSlo and/or substitutions of a single amino acid in a peptide or polypeptide sequence encoding hSlo. The substitution of a single nucleotide in a DNA, cDNA, or RNA sequence encoding hSlo and/or a single amino acid in a peptide or polypeptide sequence encoding hSlo is also referred to as a point mutation. Substitutions within a DNA, cDNA, or RNA sequence encoding hSlo and/or a peptide or polypeptide sequence encoding hSlo may be conserved or non-conserved.

Preferred modification in the hSlo gene include a point mutation at nucleic acid position 1054 when numbered in accordance with SEQ ID NO:3. This point mutation results in an amino acid substitution at position 352 of the MaxiK Channel protein when numbered in accordance with SEQ ID NO:4. For example the point mutation is a substitution of a Threonine (T) for a Serine (S) (e.g., T352S). Optionally, addition modification in the hSlo gene include point mutation that result in one or more amino acid substitution at amino acid positions 496, 602, 681, 778, 805 or 977 when numbered in accordance with SEQ ID NO:4.

Transfer of the T352S point mutation to a cell and subsequent expression of the modified MaxiK channel in that cell causes an increased current of the resultant MaxiK channel at lower intracellular calcium ion concentrations when compared to MaxiK channels encoded by the wild type sequence. The MaxiK channel encoded the T352S human BKα construct (pVAX-hSlo-T352S) is more physiologically effective than a MaxiK channel encoded by a wild type sequence or wild type sequence containing construct to treat age- and disease-induced alternations in wild-type MaxiK channel function.

To generate the T352S point mutation, wild type human BKα channel (hslo) cDNA was subcloned into the pVAX to generate pVAX-hSlo. The T352S human BKα construct (pVAX-hSlo-T352S) was prepared from pVAX-hSlo by using the QuickChange II site-directed mutagenesis kit (Agilent Technologies, Inc.) according to the manufacturer's instructions. The primers used for T352S mutation were as follows: 5′-ATGGTCACAATGTCCTCCGTTGGTTATGGGGAT-3′ (SEQ ID NO: 1) and 5′-ATCCCCATAACCAACGGAGGACATTGTGACCAT-3′ (SEQ ID NO: 2). The T352S mutation was verified by DNA sequencing. Transient transfection of HEK293 cells was performed with FuGENE® 6 (ROCHE) according to the manufacturer's instructions.

To test the effects of double point mutations on the electrical properties of the hSlo T352S channel, six separate double mutations were created. Each double point mutation was generated with the expectation that the double mutation would both inhibit the negative effect of peroxynitirite of the BK channel and increase the current state measured at low calcium. The double mutations were cytosine for adenine (C for A) and methionine for lysine (M for L) substitutions in the following constructs; pVAX-hSloT352S-C977A (C1), pVAX-hSloT352S-C496A (C2), pVAX-hSloT352S-C681A (C3), pVAX-hSloT352S-M602L (M1), pVAX-hSloT352S-M778L (M2) and pVAX-hSloT352S-M805L (M3).

The wild type human BKα channel (hslo) cDNA (SEQ ID NO: 3) and corresponding amino acid sequence (SEQ ID NO: 4, bolded) are provided below each line of nucleic acid sequence. The mutation primer (SEQ ID NO: 1, underlined) is aligned with the wild type nucleic acid sequence (SEQ ID NO: 3) from positions 1039 to 1071, with a single point mutation at position 1054 of the nucleic acid sequence (SEQ ID NO: 3), corresponding to position 352 of the amino acid sequence (SEQ ID NO: 4). This point mutation includes a substitution of a Threonine (T) for a Serine (S) at position 352 of the amino acid sequence (SEQ ID NO: 4), the T352S point mutation in both the mutation primer and resultant amino acid sequence highlighted by white lettering on a black background.

Additional mutations in the amino acid sequence (SEQ ID NO: 4) are also highlighted by white lettering on a black background and accompanied by the name of the mutation (e.g. C977A (C1), C496A (C2), C681A (C3), M602L (M1), M778L (M2) and M805L (M3)). Double mutant sequences of the disclosure may include the T352S mutation and at least one of the additional mutants provided below.

| | | |
|---|---|---|
| 1 | ATGGCAAATGGTGGCGGCGGCGGCGGCGGCAGCAGCGGCGGCGGCGGCGGCGGCGGAGGC | 60 |
| 1 | M A N G G G G G G G S S G G G G G G G G | |
| 61 | AGCAGTCTTAGAATGAGTAGCAATATCCACGCGAACCATCTCAGCCTAGACGTGTCCTCC | 120 |
| 21 | S S L R M S S N I H A N H L S L D V S S | |
| 121 | TCCTCCTCCTCCTCCTCTTCCTCTTCTTCTTCTTCCTCCTCCTCTTCCTCCTCGTCCTCG | 180 |
| 41 | S S S S S S S S S S S S S S S S S S S S | |
| 181 | GTCCACGAGCCCAAGATGGATGCGCTCATCATCCCGGTGACCATGGAGGTGCCGTGCGAC | 240 |
| 61 | V H E P K M D A L I I P V T M E V P C D | |
| 241 | AGCCGGGGCCAACGCATGTGGTGGGCTTTCCTGGCCTCCTCCATGGTGACTTTCTTCGGG | 300 |
| 81 | S R G Q R M W W A F L A S S M V T F F G | |
| 301 | GGCCTCTTCATCATCTTGCTCTGGCGGACGCTCAAGTACCTGTGGACCGTGTGCTGCCAC | 360 |
| 101 | G L F I I L L W R T L K Y L W T V C C H | |
| 361 | TGCGGGGGCAAGACGAAGGAGGCCCAGAAGATTAACAATGGCTCAAGCCAGGCGGATGGC | 420 |
| 121 | C G G K T K E A Q K I N N G S S Q A D G | |
| 421 | ACTCTCAAACCAGTGGATGAAAAAGAGGAGGCAGTGGCCGCCGAGGTCGGCTGGATGACC | 480 |
| 141 | T L K P V D E K E E A V A A E V G W M T | |
| 481 | TCCGTGAAGGACTGGGCGGGGGTGATGATATCCGCCCAGACACTGACTGGCAGAGTCCTG | 540 |
| 161 | S V K D W A G V M I S A Q T L T G R V L | |
| 541 | GTTGTCTTAGTCTTTGCTCTCAGCATCGGTGCACTTGTAATATACTTCATAGATTCATCA | 600 |
| 181 | V V L V F A L S I G A L V I Y F I D S S | |
| 601 | AACCCAATAGAATCCTGCCAGAATTTCTACAAAGATTTCACATTACAGATCGACATGGCT | 660 |
| 201 | N P I E S C Q N F Y K D F T L Q I D M A | |
| 661 | TTCAACGTGTTCTTCCTTCTCTACTTCGGCTTGCGGTTTATTGCAGCCAACGATAAATTG | 720 |
| 221 | F N V F F L L Y F G L R F I A A N D K L | |
| 721 | TGGTTCTGGCTGGAAGTGAACTCTGTAGTGGATTTCTTCACGGTGCCCCCCGTGTTTGTG | 780 |
| 241 | W F W L E V N S V V D F F T V P P V F V | |
| 781 | TCTGTGTACTTAAACAGAAGTTGGCTTGGTTTGAGATTTTTAAGAGCTCTGAGACTGATA | 840 |
| 261 | S V Y L N R S W L G L R F L R A L R L I | |
| 841 | CAGTTTTCAGAAATTTTGCAGTTTCTGAATATTCTTAAAACAAGTAATTCCATCAAGCTG | 900 |
| 281 | Q F S E I L Q F L N I L K T S N S I K L | |
| 901 | GTGAATCTGCTCTCCATATTTATCAGCACGTGGCTGACTGCAGCCGGGTTCATCCATTTG | 960 |
| 301 | V N L L S I F I S T W L T A A G F I H L | |
| 961 | GTGGAGAATTCAGGGGACCCATGGGAAAATTTCCAAAACAACCAGGCTCTCACCTACTGG | 1020 |
| 321 | V E N S G D P W E N F Q N N Q A L T Y W | |
| 1021 | GAATGTGTCTATTTACTCATGGTCACAATGTCCACCGTTGGTTATGGGGATGTTTATGCA | 1080 |

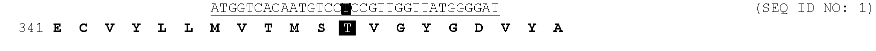

| | | |
|---|---|---|
| 1081 | AAAACCACACTTGGGCGCCTCTTCATGGTCTTCTTCATCCTCGGGGGACTGGCCATGTTT | 1140 |
| 361 | K T T L G R L F M V F F I L G G L A M F | |
| 1141 | GCCAGCTACGTCCCTGAAATCATAGAGTTAATAGGAAACCGCAAGAAATACGGGGGCTCC | 1200 |
| 381 | A S Y V P E I I E L I G N R K K Y G G S | |
| 1201 | TATAGTGCGGTTAGTGGAAGAAAGCACATTGTGGTCTGCGGACACATCACTCTGGAGAGT | 1260 |
| 401 | Y S A V S G R K H I V V C G H I T L E S | |
| 1261 | GTTTCCAACTTCCTGAAGGACTTTCTGCACAAGGACCGGGATGACGTCAATGTGGAGATC | 1320 |
| 421 | V S N F L K D F L H K D R D D V N V E I | |
| 1321 | GTTTTTCTTCACAACATCTCCCCCAACCTGGAGCTTGAAGCTCTGTTCAAACGACATTTT | 1380 |
| 441 | V F L H N I S P N L E L E A L F K R H F | |
| 1381 | ACTCAGGTGGAATTTTATCAGGGTTCCGTCCTCAATCCACATGATCTTGCAAGAGTCAAG | 1440 |
| 461 | T Q V E F Y Q G S V L N P H D L A R V K | |
| 1441 | ATAGAGTCAGCAGATGCATGCCTGATCCTTGCCAACAAGTACTGCGCTGACCCGGATGCG | 1500 |

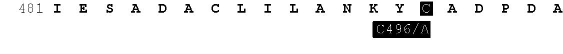

| | | |
|---|---|---|
| 1501 | GAGGATGCCTCGAATATCATGAGAGTAATCTCCATAAAGAACTACCATCCGAAGATAAGA | 1560 |
| 501 | E D A S N I M R V I S I K N Y H P K I R | |
| 1561 | ATCATCACTCAAATGCTGCAGTATCACAACAAGGCCCATCTGCTAAACATCCCGAGCTGG | 1620 |
| 521 | I I T Q M L Q Y H N K A H L L N I P S W | |
| 1621 | AATTGGAAAGAAGGTGATGACGCAATCTGCCTCGCAGAGTTGAAGTTGGGCTTCATAGCC | 1680 |
| 541 | N W K E G D D A I C L A E L K L G F I A | |
| 1681 | CAGAGCTGCCTGGCTCAAGGCCTCTCCACCATGCTTGCCAACCTCTTCTCCATGAGGTCA | 1740 |
| 561 | Q S C L A Q G L S T M L A N L F S M R S | |
| 1741 | TTCATAAAGATTGAGGAAGACACATGGCAGAAATACTACTTGGAAGGAGTCTCAAATGAA | 1800 |
| 581 | F I K I E E D T W Q K Y Y L E G V S N E | |
| 1801 | ATGTACACAGAATATCTCTCCAGTGCCTTCGTGGGTCTGTCCTTCCCTACTGTTTGTGAG | 1860 |

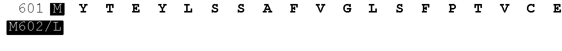

| | | |
|---|---|---|
| 1861 | CTGTGTTTTGTGAAGCTCAAGCTCCTAATGATAGCCATTGAGTACAAGTCTGCCAACCGA | 1920 |
| 621 | L C F V K L K L L M I A I E Y K S A N R | |
| 1921 | GAGAGCCGTATATTAATTAATCCTGGAAACCATCTTAAGATCCAAGAAGGTACTTTAGGA | 1980 |
| 641 | E S R I L I N P G N H L K I Q E G T L G | |
| 1981 | TTTTTCATCGCAAGTGATGCCAAAGAAGTTAAAAGGGCATTTTTTTACTGCAAGGCCTGT | 2040 |

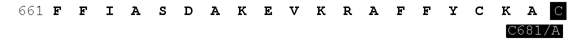

| | | |
|---|---|---|
| 2041 | CATGATGACATCACAGATCCCAAAAGAATAAAAAAATGTGGCTGCAAACGGCTTGAAGAT | 2100 |
| 681 | H D D I T D P K R I K K C G C K R L E D | |
| 2101 | GAGCAGCCGTCAACACTATCACCAAAAAAAAAGCAACGGAATGGAGGCATGCGGAACTCA | 2160 |
| 701 | E Q P S T L S P K K K Q R N G G M R N S | |
| 2161 | CCCAACACCTCGCCTAAGCTGATGAGGCATGACCCCTTGTTAATTCCTGGCAATGATCAG | 2220 |
| 721 | P N T S P K L M R H D P L L I P G N D Q | |
| 2221 | ATTGACAACATGGACTCCAATGTGAAGAAGTACGACTCTACTGGGATGTTTCACTGGTGT | 2280 |
| 741 | I D N M D S N V K K Y D S T G M F H W C | |
| 2281 | GCACCCAAGGAGATAGAGAAAGTCATCCTGACTCGAAGTGAAGCTGCCATGACCGTCCTG | 2340 |

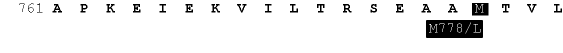

| | | |
|---|---|---|
| 2341 | AGTGGCCATGTCGTGGTCTGCATCTTTGGCGACGTCAGCTCAGCCCTGATCGGCCTCCGG | 2400 |
| 781 | S G H V V V C I F G D V S S A L I G L R | |
| 2401 | AACCTGGTGATGCCGCTCCGTGCCAGCAACTTTCATTACCATGAGCTCAAGCACATTGTG | 2460 |

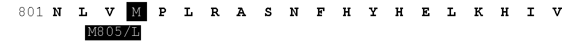

| | | |
|---|---|---|
| 2461 | TTTGTGGGCTCTATTGAGTACCTCAAGCGGGAATGGGAGACGCTTCATAACTTCCCCAAA | 2520 |
| 821 | F V G S I E Y L K R E W E T L H N F P K | |
| 2521 | GTGTCCATATTGCCTGGTACGCCATTAAGTCGGGCTGATTTAAGGGCTGTCAACATCAAC | 2580 |
| 841 | V S I L P G T P L S R A D L R A V N I N | |
| 2581 | CTCTGTGACATGTGCGTTATCCTGTCAGCCAATCAGAATAATATTGATGATACTTCGCTG | 2640 |
| 861 | L C D M C V I L S A N Q N N I D D T S L | |
| 2641 | CAGGACAAGGAATGCATCTTGGCGTCACTCAACATCAAATCTATGCAGTTTGATGACAGC | 2700 |
| 881 | Q D K E C I L A S L N I K S M Q F D D S | |
| 2701 | ATCGGAGTCTTGCAGGCTAATTCCCAAGGGTTCACACCTCCAGGAATGGATAGATCCTCT | 2760 |
| 901 | I G V L Q A N S Q G F T P P G M D R S S | |
| 2761 | CCAGATAACAGCCCAGTGCACGGGATGTTACGTCAACCATCCATCACAACTGGGGTCAAC | 2820 |
| 921 | P D N S P V H G M L R Q P S I T T G V N | |
| 2821 | ATCCCCATCATCACTGAACTAGTGAACGATACTAATGTTCAGTTTTTGGACCAAGACGAT | 2880 |
| 941 | I P I I T E L V N D T N V Q F L D Q D D | |
| 2881 | GATGATGACCCTGATACAGAACTGTACCTCACGCAGCCCTTTGCCTGTGGGACAGCATTT | 2940 |

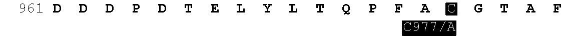

| | | |
|---|---|---|
| 2941 | GCCGTCAGTGTCCTGGACTCACTCATGAGCGCGACGTACTTCAATGACAATATCCTCACC | 3000 |
| 981 | A V S V L D S L M S A T Y F N D N I L T | |
| 3001 | CTGATACGGACCCTGGTGACCGGAGGAGCCACGCCGGAGCTGGAGGCTCTGATTGCTGAG | 3060 |
| 1001 | L I R T L V T G G A T P E L E A L I A E | |
| 3061 | GAAAACGCCCTTAGAGGTGGCTACAGCACCCCGCAGACACTGGCCAATAGGGACCGCTGC | 3120 |
| 1021 | E N A L R G G Y S T P Q T L A N R D R C | |
| 3121 | CGCGTGGCCCAGTTAGCTCTGCTCGATGGGCCATTTGCGGACTTAGGGGATGGTGGTTGT | 3180 |
| 1041 | R V A Q L A L L D G P F A D L G D G G C | |
| 3181 | TATGGTGATCTGTTCTGCAAAGCTCTGAAAACATATAATATGCTTTGTTTTGGAATTTAC | 3240 |
| 1061 | Y G D L F C K A L K T Y N M L C F G I Y | |
| 3241 | CGGCTGAGAGATGCTCACCTCAGCACCCCCAGTCAGTGCACAAAGAGGTATGTCATCACC | 3300 |
| 1081 | R L R D A H L S T P S Q C T K R Y V I T | |
| 3301 | AACCCGCCCTATGAGTTTGAGCTCGTGCCGACGGACCTGATCTTCTGCTTAATGCAGTTT | 3360 |
| 1101 | N P P Y E F E L V P T D L I F C L M Q F | |
| 3361 | GACCACAATGCCGGCCAGTCCCGGGCCAGCCTGTCCCATTCCTCCCACTCGTCGCAGTCC | 3420 |
| 1121 | D H N A G Q S R A S L S H S S H S S Q S | |
| 3421 | TCCAGCAAGAAGAGCTCCTCTGTTCACTCCATCCCATCCACAGCAAACCGACAGAACCGG | 3480 |
| 1141 | S S K K S S S V H S I P S T A N R Q N R | |
| 3481 | CCCAAGTCCAGGGAGTCCCGGGACAAACAGAAGTACGTGCAGGAAGAGCGGCTT | 3538 (SEQ ID NO: 3) |
| 1161 | P K S R E S R D K Q K Y V Q E E R L | (SEQ ID NO: 4) |

### METHODS OF THE INVENTION

The present invention provides a method of gene therapy for treating physiological dysfunctions of smooth muscle. Physiological dysfunctions of smooth muscle include for example, over active bladder (OAB) syndrome, erectile dysfunction (ED), asthma; benign hyperplasia of the prostate gland (BHP); coronary artery disease (infused during angiography); genitourinary dysfunctions of the bladder, endopelvic fascia, prostate gland, ureter, urethra, urinary tract, and vas deferens; irritable bowel syndrome; migraine headaches; premature labor; Raynaud's syndrome; and thromboangitis obliterans.

OAB syndrome is characterized by a group symptoms that include, but are not limited to, urinary urgency, frequency, nocturia and incontinence is both a common and significant medical problem that affects over 17% of men and women in the United States. OAB, with and without incontinence, has a clinically significant impact on quality of life, quality of sleep, and mental health, in both men and women. Despite the adverse effects on quality of life, as many as 40% of the people with OAB do not discuss it with their physician or healthcare professional. Of those that do mention it, only 25% may receive pharmacological therapy. Commonly prescribed FDA approved treatments of choice for OAB/UUI include the non-selective oral muscarinic receptor antagonists (e.g. oxybutynin, tolterodine), the recently approved β₃-adrenoceptor agonist Mirabegron, and the direct injected OnabotulinumtoxinA (BOTOX). The oral medications are associated with dose-related side effects such as dry mouth, dry eye, constipation and the injectable with urinary retention. Newer and more selective agents also display these adverse problems. None of the existing therapies are effective at dosages that do not have those disabling side effects. The combination of lack of efficacy and a significant side effect profile greatly reduces the long term use of these medications by patients (e.g. non-selective oral muscarinic receptor antagonists, β₃-adrenoceptor agonists, and OnabotulinumtoxinA. Consequently, there is a significant need to develop new therapies that can effectively treat OAB in those millions of afflicted people without deleterious or disabling side effects.

The degree of contraction of detrusor smooth muscle is critical to the storage and emptying function(s) of the bladder. Potassium (K⁺) channels play an important role in this process by virtue of their ability to alter the membrane potential and excitability of smooth muscle cells. As with many other smooth muscle cell types, K⁺ channels play a critical role in the modulation of detrusor myocyte tone. A number of distinct K+ channel subtypes have been identified in detrusor myocytes from various species. The central role played by K⁺ channels in modulating bladder function may arise from their functionally antagonistic relationship with transmembrane calcium flux through voltage dependent calcium channels. This hypothesis is based, at least in part, upon the discovery that MaxiK knock-out mice exhibit bladder dysfunction.

Approximately 30 million men are affected by ED in the United States. Existing therapies have deleterious side effects. The use of phosphodiesterase type 5 (PDE5) inhibitors has a success rate of only 60%. Surgical implants to treat ED cost in excess of $20,000 for the device and surgical procedures. Furthermore, existing therapies require ED patients to plan for sexual intercourse.

Exemplary, smooth muscle cells for which the present method of gene therapy may be used include, but are not limited to, visceral smooth muscle cells of the bladder, bowel, bronchi of the lungs, penis (corpus cavernosum), prostate gland, ureter, urethra (corpus spongiosum), urinary tract, and vas deferens, as well as the smooth and/or skeletal muscle cells of the endopelvic fascia. Specifically, the claimed method of gene therapy may be used in bladder smooth muscle cells, colonic smooth muscle cells, corporal smooth muscle cells, gastrointestinal smooth muscle cells, prostatic smooth muscle, and urethral smooth muscle. Given the many gross histological and physiological similarities in the factors that regulate the tone of smooth muscle tissue and of other vascular tissue, it follows naturally that similar principles would permit the application of the present method of gene therapy to the arterial smooth muscle cells of the bladder, bowel, bronchi of the lungs, penis (corpus cavernosum), prostate gland, ureter, urethra (corpus spongiosum), urinary tract, and vas deferens.

The nucleic acid sequence of interest may be introduced into a smooth muscle cell by a number of procedures known to one skilled in the art, such as electroporation, DEAE Dextran, monocationic liposome fusion, polycationic liposome fusion, protoplast fusion, DNA-coated microprojectile bombardment, creation of an in vivo electrical field, injection with recombinant replication-defective viruses, homologous recombination, nanoparticles, and naked DNA transfer by, for example, intravesical instillation. It is to be appreciated by one skilled in the art that any of the above methods of DNA transfer may be combined.

In preferred embodiments, a mutated hSlo gene is encapsulated in nanoparticles and administered for example by installation into the lumen of bladder.

Alternatively, the mutated hSlo gene is transferred into the smooth muscle cells by naked DNA transfer, using a mammalian vector. “Naked DNA” is herein defined as DNA contained in a non-viral vector. The DNA sequence may be combined with a sterile aqueous solution, which is preferably isotonic with the blood of the recipient. Such a solution may be prepared by suspending the DNA in water containing physiologically-compatible substances (such as sodium chloride, glycine, and the like), maintaining a buffered pH compatible with physiological conditions, and rendering the solution sterile. In a preferred embodiment of the invention, the DNA is combined with a 20-25% sucrose-in-saline solution, in preparation for introduction into a smooth muscle cell.

The mutated hSlo gene is transferred into the smooth muscle cells by an adenoviral vector containing the mutated hSlo gene.

Where the naked DNA, nanoparticle, or adenoviral vector is transferred into smooth muscle cells of the bladder, it is introduced into the bladder by intravesical instillation of a solution of naked DNA, nanoparticles, or adenoviral vectors.

The solution is then voluntarily withheld by the patient, within the bladder, for a prescribed duration of time. In another embodiment, solution of naked DNA, nanoparticles, or adenoviral vector is introduced into the endopelvic fascia, prostate, ureter, urethra, upper urinary tract, or vas deferens by instillation or injection therapy, and the ureter, urethra, or upper urinary tract is obstructed so that the solution remains in contact with the internal epithelial layer for a prescribed period of time. The mutated hSlo gene for expression may also be directly injected into the smooth muscle cells of the subject.

The present invention specifically provides a method of gene therapy wherein the mutated protein, i.e, mutated MaxiK channel protein involved in the regulation of smooth muscle tone modulates relaxation of smooth muscle. These proteins will enhance relaxation of smooth muscle, and will also decrease smooth muscle tone. In particular, where vasorelaxation is enhanced in penile smooth muscle, an erection will be more easily attained.

Similarly, where smooth muscle tone is decreased in the bladder, bladder capacity will be increased. In this embodiment of the invention, the gene therapy method is particularly useful for treating individuals with bladder hyperreflexia. As used herein, a “hyperreflexic bladder” is one which contracts spontaneously so that an individual is unable to control the passage of urine. This urinary disorder is more commonly called urge incontinence, and may include urge incontinence combined with stress incontinence.

It is to be understood that the method of gene therapy described by the present invention may involve the transfer into a smooth muscle cell, whose function is involved in contraction and/or relaxation, of more than one nucleic acid sequence encoding a protein.

The present invention specifically provides a method of regulating penile smooth muscle tone in a subject, comprising the introduction, into penile smooth muscle cells of the subject, of a DNA sequence encoding a protein involved in the regulation of smooth muscle tone, and expression in a sufficient number of penile smooth muscle cells of the subject to induce penile erection in the subject. In this embodiment, the method of the present invention is used to alleviate erectile dysfunction. The erectile dysfunction may result from a variety of disorders, including neurogenic, arteriogenic, and veno-occlusive dysfunctions, as well as other conditions which cause incomplete relaxation of the smooth muscle. The subject may be animal or human, is preferably human.

Furthermore, the present invention specifically provides a method of regulating bladder smooth muscle tone in a subject, comprising the introduction, into bladder smooth muscle cells of the subject, of a DNA sequence encoding a protein involved in the regulation of smooth muscle tone, and expression in a sufficient number of bladder smooth muscle cells of the subject to enhance bladder relaxation in the subject. In this embodiment, the method of the present invention is used to alleviate a hyperreflexic bladder. A hyperreflexic bladder may result from a variety of disorders, including neurogenic and arteriogenic dysfunctions, as well as other conditions which cause incomplete relaxation or heightened contractility of the smooth muscle of the bladder. The subject may be animal or human, and is preferably human.

In other embodiments of the invention, the method of gene therapy described herein is used to treat other dysfunctions relating to the performance of smooth muscle, including, but not limited to, asthma; coronary artery disease (infused during angiography); genitourinary dysfunctions of the ureter, urethra, urinary tract, and vas deferens; irritable bowel syndrome; migraine headaches; premature labor; Raynaud's syndrome; and thromboangitis obliterans. When used to treat asthma, the present method of gene therapy may be administered to a subject by way of aerosol delivery using any method known in the art.

The present invention also provides viral and non-viral recombinant vectors and plasmids. A viral-based vector comprises: (1) nucleic acid of, or corresponding to at least a portion of, the genome of a virus, which portion is capable of directing the expression of a DNA sequence; and (2) a DNA sequence encoding a protein involved in the regulation of smooth muscle tone, operably linked to the viral nucleic acid and capable of being expressed as a functional gene product in the target cell. The recombinant viral vectors of the present invention may be derived from a variety of viral nucleic acids known to one skilled in the art, e.g., the genomes of adenovirus, adeno-associated virus, HSV, Semiliki Forest virus, vaccinia virus, and other viruses, including RNA and DNA viruses.

The recombinant vectors and plasmids of the present invention may also contain a nucleotide sequence encoding suitable regulatory elements, so as to effect expression of the vector construct in a suitable host cell. As used herein, “expression” refers to the ability of the vector to transcribe the inserted DNA sequence into mRNA so that synthesis of the protein encoded by the inserted nucleic acid can occur. Those skilled in the art will appreciate the following: (1) that a variety of enhancers and promoters are suitable for use in the constructs of the invention; and (2) that the constructs will contain the necessary start, termination, and control sequences for proper transcription and processing of the DNA sequence encoding a protein involved in the regulation of smooth muscle tone, upon introduction of the recombinant vector construct into a host cell.

The non-viral vectors provided by the present invention, for the expression in a smooth muscle cell of the DNA sequence encoding a protein involved in the regulation of smooth muscle tone, may comprise all or a portion of any of the following vectors known to one skilled in the art: pCMVβ (Invitrogen), pcDNA3 (Invitrogen), pET-3d (Novagen), pProEx-1 (Life Technologies), pFastBac 1 (Life Technologies), pSFV (Life Technologies), pcDNA2 (Invitrogen), pSL301 (Invitrogen), pSE280 (Invitrogen), pSE380 (Invitrogen), pSE420 (Invitrogen), pTrcHis A,B,C (Invitrogen), pRSET A,B,C (Invitrogen), pYES2 (Invitrogen), pAC360 (Invitrogen), pVL1392 and pV11392 (Invitrogen), pCDM8 (Invitrogen), pcDNA I (Invitrogen), pcDNA I(amp) (Invitrogen), pZeoSV (Invitrogen), pRc/CMV (Invitrogen), pRc/RSV (Invitrogen), pREP4 (Invitrogen), pREP7 (Invitrogen), pREP8 (Invitrogen), pREP9 (Invitrogen), pREP10 (Invitrogen), pCEP4 (Invitrogen), pEBVHis (Invitrogen), and λPop6. Other vectors would be apparent to one skilled in the art.

Promoters suitable for the present invention include, but are not limited to, constitutive promoters, tissue-specific promoters, and inducible promoters. Preferably, the promotor is not an urothelium specific expression promotor.

In one embodiment of the invention, expression of the DNA sequence encoding a protein involved in the regulation of smooth muscle tone is controlled and affected by the particular vector into which the DNA sequence has been introduced. Some eukaryotic vectors have been engineered so that they are capable of expressing inserted nucleic acids to high levels within the host cell. Such vectors utilize one of a number of powerful promoters to direct the high level of expression. Eukaryotic vectors use promoter-enhancer sequences of viral genes, especially those of tumor viruses. This particular embodiment of the invention provides for regulation of expression of the DNA sequence encoding the protein, through the use of inducible promoters. Non-limiting examples of inducible promoters include metallothionine promoters and mouse mammary tumor virus promoters. Depending on the vector, expression of the DNA sequence in the smooth muscle cell would be induced by the addition of a specific compound at a certain point in the growth cycle of the cell. Other examples of promoters and enhancers effective for use in the recombinant vectors of the present invention include, but are not limited to, CMV (cytomegalovirus), SV40 (simian virus 40), HSV (herpes simplex virus), EBV (Epstein-Barr virus), retrovirus, adenoviral promoters and enhancers, and smooth-muscle-specific promoters and enhancers. An example of a smooth-muscle-specific promoter is SM22α.

The present invention further provides a smooth muscle cell which expresses an exogenous DNA sequence encoding a protein involved in the regulation of smooth muscle tone. As used herein, “exogenous” means any DNA that is introduced into an organism or cell.

The introduction into the smooth muscle cell of a recombinant vector or plamid containing the exogenous DNA sequence may be effected by methods known to one skilled in the art, such as electroporation, DEAE Dextran, cationic liposome fusion, protoplast fusion, DNA-coated microproj ectile bombardment, injection with recombinant replication-defective viruses, homologous recombination, nanoparticles, and naked DNA transfer by, for example, intravesical instillation. It is to be appreciated by one skilled in the art that any of the above methods of DNA transfer may be combined. In preferred embodiments, the vector or plasmid containing a mutated hSlo gene is encapsulated in nanoparticles and administered for example by installation into the lumen of bladder,

Nanoparticle Delivery System

Compositions of the disclosure may be administered to a subject in need using a variety of methods, including, but not limited to, organ instillation and injection. In some embodiments, organ installation is preferred. When used to treat over active bladder (OAB) syndrome, compositions of the disclosure may be installed within the bladder. When used to treat erectile dysfunction (ED), compositions of the disclosure may be installed within the penis.

Delivery platforms that can deliver a DNA or cDNA plasmid of the disclosure efficiently across the urothelial barrier to treat OAB (or dermal barrier(s) to treat ED) increase the efficiency of gene transfer in the bladder (or penis). For example, nanoparticles are engineered to deliver nucleic acids. Preferably, nanoparticles are engineered to deliver the hMaxiK gene therapy vectors of the disclosure.

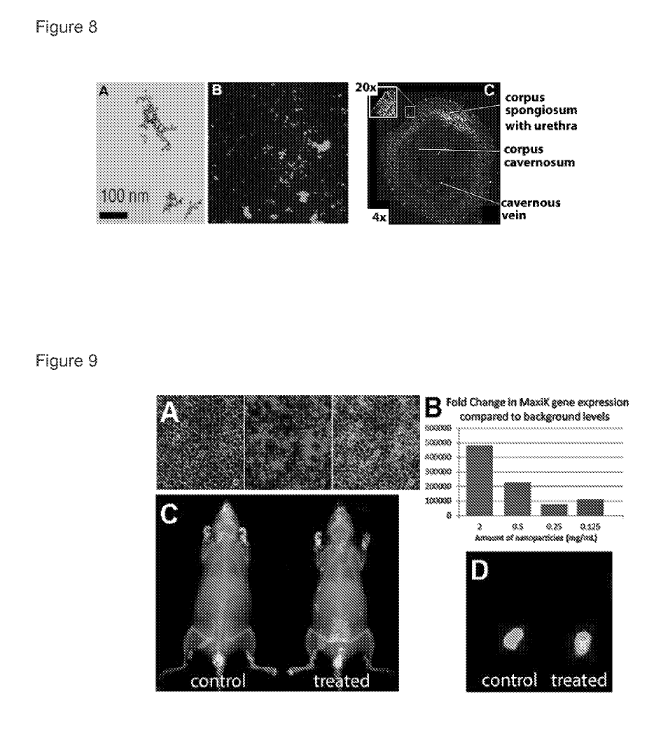

Compositions and methods of the disclosure may include a biocompatible nanoparticle platform having intrinsic plasticity to enable the user to chemically tune both the internal (e.g. hydrophobicity, charge) and external (e.g. surface charge, PEGylation) properties. The material of the biocompatible nanoparticle platform may be converted into powders composed of nanoparticles with average diameters of about 10 to about 99 nanometers (nm) (FIG. 8). Powders composed of nanoparticles can deliver specific concentrations of encapsulated therapeutic product over extended time periods. This platform can deliver bioactive molecules both systemically and topically. No indications of induced inflammation or toxicity have been observed. Appreciable cell uptake of the nanoparticles occurs without cytotoxicity. Following uptake, nanoparticles release functional DNA.

Nanoparticles may be tuned to accommodate a wide range of biomolecules by manipulating the internal charge and hydrophobicity through the use of dopant trimethoxysilanes with the fourth site having the desired chemical moiety (e.g. alkyl or amine groups), in lieu of the fourth methoxy group that is present in the basic building block for the nano platform-tetramethoxysilane (TMOS). TMOS particles contacted with silanes having positive charge (amines) are contemplated for plasmid encapsulation.

Topical delivery offers several other advantages over other routes of administration (oral or injection) with regards to target specific impact, decreased systemic toxicity, avoidance of first pass metabolism, variable dosing schedules, and broadened utility to diverse patient populations. Chemical penetration enhancers may be used in order to perturb the epidermal barrier (e.g. membrane keratin and lipid bilayer). However, preliminary pathology studies on animals suggest there is no acute pathology associated with the nanoparticles of this disclosure.

The urothelium of the bladder has evolved mechanisms to impede exogenous molecules from passage. Consequently, topical bladder therapy has a unique and advantageous set of physiologic attributes that circumvent the challenge of traversing the urothelium. Thus, the nanoparticles of the disclosure demonstrate the superior property of increased efficiency in crossing the urothelium barrier, a characteristic that is particularly advantageous when the nanoparticles are used to treat over active bladder (OAB) syndrome.

### EXAMPLES

### Example 1

### General Methods

Animal Model of Bladder Overactivity:

Although there is no animal model that completely recapitulates all aspects of the corresponding human condition, the partial urethral obstruction (PUO) model to cause detrusor overactivity (DO) in the rat (the same animal model proposed herein) is generally accepted in the peer reviewed literature and by the NIH. Furthermore this animal model was used by ICI to support their successful IND application for MaxiK treatment for the OAB indication by the FDA.^{13,35-40} Female Sprague-Dawley (250 g) rats will be used in this study. PUO will be induced as previously published by us. (11) Briefly, the urethra will be isolated, a sterile metal bar with a diameter of 0.91 mm will be placed on the urethral surface, and a 3-0 silk suture tied around both the urethra and the bar. When the suture is secured, the bar is removed, leaving the urethra partially obstructed. The abdominal muscle layer and skin are then closed. Controls (sham) will undergo the same surgical procedure, except for tying of the suture around the urethra.

Suprapubic Bladder Catheterization:

A second surgical procedure will be done on all rats 2 weeks after the PUO procedure. A lower abdominal and perineal midline incision will be made, the bladder will be exposed, the obstructing urethral silk suture will be removed, a small incision will be made in bladder dome and a cuffed polyethylene cannula will be inserted into the bladder and secured with a purse string suture. The cannula will then be tunneled through the subcutaneous space and exited through an incision on the back of the animal's neck, closed and secured with sutures. To prevent infections, all rats will receive an injection of sulfadoxin (24 mg/kg) and trimethoprim (4.8 mg/kg) subcutaneously.

Cystometry:

Cystometric studies will be performed in unrestrained rats 48 hours after bladder catheterization and removal of urethral obstruction (baseline measurements), and 48 hrs after intravesical treatment with nanoparticles. Cystometry will be performed as previously described by us.^{18,37,40} Briefly, the animals will be placed in a metabolic chamber and the indwelling bladder catheter will be connected to a two-way valve and attached to a pressure transducer and an infusion pump. The pressure transducer will be connected via a transducer amplifier (ETH 400 CB Sciences) to a data-acquisition board (MacLab/8e, ADI Instruments). Real-time display and recording of pressure measurements will be done on a Macintosh computer (MacLab software, version 3.4, ADI Instruments). The pressure transducers will be calibrated (in cmH2O) before each experiment. The rate of bladder infusion will be set at 1.5 mL/min using a programmable Harvard infusion pump (model PHD 2000). Cystometric activity will be continuously recorded after the first micturition and subsequently for at least ten additional reproducible micturition cycles; as micturitions occur ˜20 min apart, at least 1.5 h of data will be recorded from each animal. Relevant urodynamic parameters will then be quantified offline from each cystometrogram (see details below) as previously described.^{18,37,40}

Intravesical Administration of Naked Plasmid and Nanoparticle Encapsulating Plasmid:

One hour after cystometric evaluation (acquisition of baseline measurements) the animals will be anesthetized with isoflurane, the bladder emptied by massaging the pelvic region, and the naked plasmid or the nanoparticle encapsulating plasmid will be injected in the bladder lumen through the bladder indwelling catheter. The plasmid and nanoparticles will be reconstituted in sterile 0.9% saline and 200 uL of the desired concentration will be injected, followed by 100 μL of saline only to account for the 50 μL catheter “deadspace”.

Evaluation of Bladder Function:

Bladder function will be evaluated based on the following urodynamic parameters: 1) bladder capacity, the volume of infused saline at micturition; 2) basal pressure, the lowest bladder pressure recorded during cystometry between voiding; 3) threshold pressure, the bladder pressure immediately before micturition; 4) micturition pressure, the peak bladder pressure during micturition; 5) micturition volume, the volume of urine discharged during micturition; 6) residual volume, the volume of infused saline minus the micturition volume for each void; and 7) spontaneous activity (SA)=mean intermicturition pressure (IMP) minus mean basal pressure (BP), an approximate index of spontaneous bladder contraction between micturitions. The IMP is the average pressure recorded between micturitions. The mean value of BP is subtracted from the mean IMP to obtain a single SA of 6 to 8 voids during a study. As such, the SA serves as an index of the fluctuations in bladder pressure, if any, between the recorded micturition reflexes, a measure of DO, and a presumptive clinical correlate of urinary urgency and a measure of response to gene transfer.^{14,35}

Ex Vivo Evaluation of Changes in Detrusor Function Induced by Treatment with hSlo and hSlo T352S:

Effects on detrusor contractility and excitability will be determined by organ bath and electrophysiology (patch clamping) in a similar manner as described for preliminary data (see FIG. 7). In order to perform these evaluations, after cystometry bladders will be harvested and cut in half, from the dome to the neck. One half will be further cut into strips that will be used in the organ bath studies, while the other half will be used to isolate detrusor smooth muscle cells for electrophysiological studies. Organ bath: Bladder strips will be mounted in organ baths at 1.0 g resting tension and spontaneous phasic contractions will be recorded with a force transducer as previously described by us (²²; see FIG. 7 E,F). Experiments will be performed in the absence and presence of iberiotoxin (IBTX; 300 nM), a MaxiK channel blocker, to evaluate the relative contribution of MaxiK channel activity to development of detrusor spontaneous activity. Electrophysiology: detrusor smooth muscle cells (SMCs) will be isolated and single cell patch-clamping recordings will be performed, as previously described (⁴¹⁻⁴³ FIG. 7H), in the absence and presence of IBTX to determine the overall contribution of MaxiK to changes in detrusor excitability.

### Example 2

### Generation of the T352S Human BKa Construct (pVAX-hSlo-T352S)

Modifications of the hSlo gene can be used to effectively treat human disease that is caused, for example, by alterations of the BK channel by age and disease.

The human BKα channel (hslo) cDNA was subcloned into the pVAX to generate pVAX-hSlo. The T352S human BKα construct (pVAX-hSlo-T352S) was prepared from pVAX-hSlo by using the QuickChange II site-directed mutagenesis kit (Agilent Technologies, Inc.) according to the manufacturer's instructions. The primers used for T352S mutation were as follows: 5′-ATGGTCACAATGTCCTCCGTTGGTTATGGGGAT-3′ (SEQ ID NO: 1) and 5′-ATCCCCATAACCAACGGAGGACATTGTGACCAT-3′ (SEQ ID NO: 2). The T352S mutation was verified by DNA sequencing. Transient transfection of HEK293 cells was performed with FuGENE® 6 (ROCHE) according to the manufacturer's instructions. The HEK cells were studied with electrophysiological patch clamp analysis under the following conditions: Currents were recorded with whole-cell patch-clamp at room temperature. Borosilicate glass electrodes had 4 to 20 MΩ tip resistances when filled with internal solution. The extracellular solution was composed of 137 mM NaCl, 5.4 mM KCl, 1 mM MgCl2, 1 mM CaCl2, 2.3 mM NaOH, 5 mM HEPES and 10 mM dextrose (pH 7.4 with NaOH). Internal solution contained 120 mM K-aspartate, 3 mM Na2ATP, 5 mM HEPES, and 5 mM EGTA (pH 7.2 with KOH). Currents were elicited with a holding potential of −80 mV with 200 ms duration testing pulses from −60 mV to +110 mV in 10 mV increments.

Clampfit (Molecular Devices, Sunnyvale, Calif., USA) and GraphPad Prism (GraphPad Software, San Diego, Calif., USA) were used for data analysis. Data are presented as mean±SEM. P<0.05 by two-way ANOVA (for comparison among groups) or Student's t-test (for comparison of individual voltage steps) was considered to indicate statistical significance.

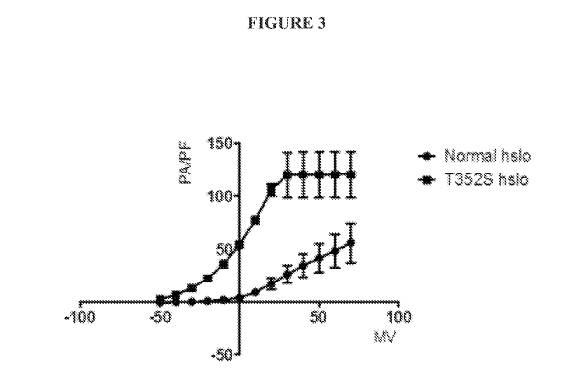

The result of the T352S site-directed mutagenesis demonstrates a leftward shift in the voltage-dependent activation curve, as shown in FIG. 3.

To test the effects of double point mutations on the electical properties of the hSlo T352S channel, six separate double mutations were created. Each double point mutation was generated with the expectation that the double mutation would both inhibit the negative effect of peroxynitirite of the BK channel and increase the current state measured at low calcium. The double mutations were cytosine for adenine (C for A) and methionine for lysine (M for L) substitutions in the following constructs; pVAX-hSloT352S-C977A (C1), pVAX-hSloT352S-C496A (C2), pVAX-hSloT352S-C681A (C3), pVAX-hSloT352S-M602L (M1), pVAX-hSloT352S-M778L (M2) and pVAX-hSloT352S-M805L (M3).

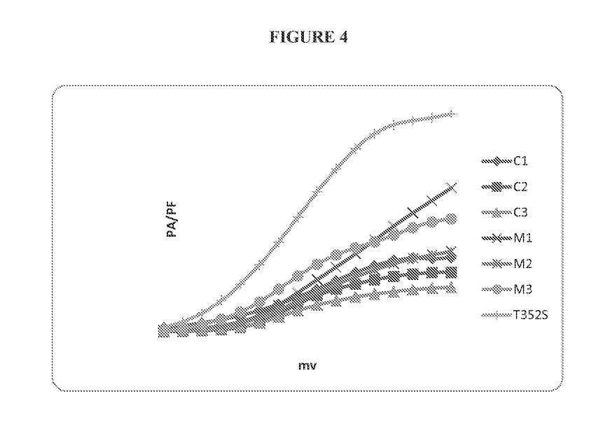

Electrophysiological patch clamp analysis of these substitution constructs was performed after transfection into HEK cells for 24-48 h in a high glucose (22.5 mM) environment. Although the T352S single point mutation is resistant to oxidative stress, the double point mutations (C1, C2, C3, M1, M2, and M3) appear to compromise the effect of the T352S single point mutation in a high glucose environment. The results of those patch clamp experiments are shown in FIG. 4.

### Example 3

### Evaluation of Newly Designed Vectors Expressing hSlo Gene T352S Will More Effectively and Safely Treat OAB when Compared to Vectors Expressing the Original Wild Type hSlo Gene

Previous studies by our group in rats with bladder overactivity created by PUO have shown that the transfection of plasmid expressing MaxiK (pVAX-hSlo) can ameliorate and, in some cases, virtually normalize many characteristics of detrusor overactivity in this animal model.³⁶ Those studies were extended to a human trial in 20 women with OAB and the results at the doses studied showed safety and some potential efficacy to treat OAB, although with more restricted efficacy than observed in our preclinical studies in the rat PUO model. In this Aim we will use the PUO rat model to determine whether the beneficial effects of intravesical treatment of DO with pVAX-hSlo can be improved by using a vector expressing a hSlo mutant (T352S) that encodes a MaxiK channel with higher sensitivity to calcium (pVAX-hSlo T352S) (FIG. 3 and ⁴⁴).

The study is designed to test activity of the gene at the half log dose concentration (0, 10, 30, and 100 μg) to allow the determination of the lowest effective dose. Vectors expressing genes from the CMV (pVAX) and the smooth muscle alpha actin (pSMAA) promoters will be tested. An estimated total of 172 rats will be used, as indicated in the Table 1.

The effects of intravesical treatment of PUO rats with control empty vectors, and with hSlo and hSlo T352S driven by the CMV and SMAA promoters will be evaluated by cystometry (see General Methods) and compared among groups (see Table 1). At conclusion of cystometric evaluations the animals will be euthanized and the bladders harvested to be used in the organ bath and electrophysiology studies (see General Methods) that will determine the effect of each treatment on overall detrusor contractility and SMC excitability.

Rationale and preliminary data: Isolated bladder strips from patients with OAB and from animal models of DO show increased spontaneous phasic contractions⁴⁵⁻⁴⁹. Potassium channels appear to play a role in the development and regulation of these phasic contractions, with decreased activity of the MaxiK channel being implicated in greater spontaneous activity⁴⁹⁻⁵². Our previous studies using the streptozotocin (STZ) Type 1 diabetic model of bladder overactivity further support the involvement of MaxiK in this phenomenon. As shown in FIG. 7, cystometric studies of STZ rats indicate the characteristically higher voiding frequencies and hyperactive bladder pressures (FIG. 4 A-D)40 and organ bath studies demonstrate that bladder strips isolated from the same animal present increase phasic activity (FIG. 7 E).⁵³⁻⁵⁵ In FIG. 7F, we show that treatment with the MaxiK inhibitor, iberiotoxin (IBTX) a specific inhibitor of MaxiK channels increases the amplitude of these phasic contractions. However, this effect is lower in strips isolated from the diabetic animal, presumably because of lower activity of the MaxiK activity in the diabetic bladder. This prediction is supported by electrophysiological studies using a standard single whole cell patch technique to look for the functional expression of these channels (FIG. 7 H)⁴¹⁻⁴³. Stepwise application of voltage across the cell membrane results in opening of channels and outward current flow. Recordings were made from detrusor cells isolated from 5 animals in triplicate. There was no significant difference between the outward current and applied voltage between cells isolated from STZ-diabetic animals with bladder hyperactivity and control rats. However, after addition of IBTX there was a greater decrease (>50%) in the response to the applied voltage in control compared with diabetic detrusor cells (FIG. 7H) supporting a reduction in the activity of the MaxiK channels in the bladder detrusor muscle of diabetic animals.

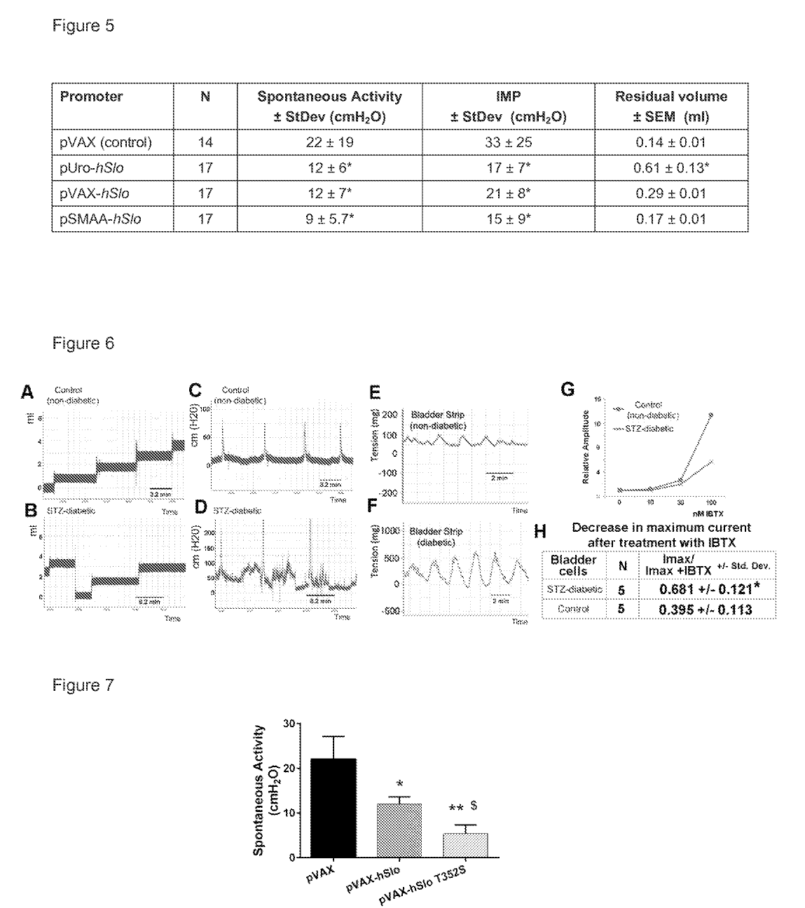

In our previous studies we observed that cystometric evaluation of PUO rats (similar to STZ rats) demonstrated a higher level of bladder spontaneous activity, a correlate for DO. Treatment with pVAX-hSlo and pSMAA-hSlo significantly ameliorated DO in these animals (see FIG. 5). Our initial cystometry studies with PUO rats treated with 30 μg of pVAX-hSlo T352S indicate that when compared to our previous data (FIG. 5) this hSlo mutant may be more efficient in reducing DO than the wild type gene (FIG. 7). Based on this preliminary finding and the characteristic properties of the mutated MaxiK channel (see FIG. 3), we expect that the mutant hSlo gene will provide a more efficient and attractive product to treat OAB.

Direct effects of hSlo and hSlo T352S expression in PUO detrusor contractility and excitability still need to be determined. However, based on our preliminary cystometric findings of reduced bladder spontaneous activity in hSlo treated animals, and from our studies with the STZ model of DO demonstrating the close association of bladder overactivity with decreased MaxiK expression, we expect to find that spontaneous phasic contractions of isolated bladder strips from PUO treated rats will be significantly lower compared to bladder strips isolated from untreated PUO animals, and more sensitive to IBTX blockade, reflecting the increased MaxiK expression (i.e. rescue of expression) in PUO detrusor.

Statistics:

Distributions of all continuous variables will be examined for normality. Those not normally distributed will be transformed using a log scale and by experience the transformations have been found to be reasonably normal. One-way analyses of variance will be performed to determine the overall significance of differences among groups, and a Duncan's multiple comparison procedure will be used to assess the significance of pair wise differences among groups. The overall level of significance will be set a priori at α=0.05.

TABLE 1
Number of animals per experimental group and doses for
intravesical treatment with empty vectors (pVAX and pSMAA)
and vectors expressing hSlo and hSlo T352S (pVAX-hSlo,
pVAK-hSlo T352S, pSMAA-hSlo and pSMAA-hSlo T352S).

Dose (μg)

| | | | |
|---|---|---|---|
| 0 | 10 | 30 | 100 |

| | |
|---|---|
| Experimental groups | Number of animals |

| | | | | |
|---|---|---|---|---|
| pVAX (control) | 10 | | | |
| PVAX-hSlo | | 27 | 27 | 27 |
| PVAX-hSlo T352S | | 27 | 27 | 27 |
| pSMAA (control) | 10 | | | |
| pSMAA-hSlo | | 27 | 27 | 27 |
| pSMAA-hSlo T352S | | 27 | 27 | 27 |

### Example 4

### Generation of Nanoparticles Carrying hSlo Expression Vectors

Basic Protocol for Preparation of Hydrogel/Glass Composites:

Tetramethoxysilane (TMOS, 5 mL) is mixed with an HCl solution (560 μl of 0.2 mM HCl added to 600 μl of deionized water) and then immediately sonicated for 45 minutes in a cool water bath after which the mixture is placed on ice. D-glucose is then added to the solutions at 40 mg glucose/mL of buffered sodium nitrite solution. After the glucose has dissolved, polyethylene glycol (PEG) 400 is then added at a ratio of 1 mL PEG/20 mL of buffered solution. Chitosan [5 mg of chitosan/mL acidified distilled water (with 1 M HCl) pH 4.5] is then added at a ratio of 1 mL chitosan solution/20 mL of buffered solution. After the buffered solution is well stirred, the previously sonicated TMOS is slowly introduced at a ratio of 2 mL TMOS/20 mL buffer. The combined mixture is then stirred immediately and set aside. The resulting mixture gels within 1-2 hours. These monolith (block) sol-gels samples are then taken out of their containers and crudely dried by blotting with paper towels prior to either heating or lyophilization. Several control samples were made with the same overall protocol, but with some lacking a specific individual component such as nitrite, glucose, chitosan and PEG. For example, an NO-free “empty gel” was made by withholding nitrite, i.e. incorporating only glucose, chitosan, and PEG.

Preparation of Heat Treated Hydrogel/Glass Composites:

The sample was heated in a closed convection oven at 70° C. until the gel became a hard, white, glassy material (24-48 hours). Excessive heating resulted in a brown discoloration indicative of carmelization of the sugar. Carmelization was never observed when the sample was heated at temperature at or below 70° C. Discolored materials were discarded. The material is then placed in a planetary ball mill (Fritsch, “Pulverisette 6”) for 60 minutes at a speed of 140 rpm.

Preparation of Lyophilized Hydrogel/Glass Composites:

The hydrogel monoliths generated using the above described protocols were placed into lyophilization flasks and lyophilized for 24 hours. The resulting material is a mix of coarse and fine white particulate matter. This mixture is then ground with a mortar and pestle resulting in a fine white powder.

Preparative Protocols for Nanoparticles Containing the hSlo Vectors.

These protocols yield a fine powder comprised of a relatively uniform distribution of nano-sized or nano-scale particles that are capable of sustained release of pVAX-hSlo when exposed to an aqueous environment.

Hydrogel monoliths of varying thicknesses may be air dried, crushed, and then heated as described above. The resulting powder may be further ground using a ball mill for varying time periods. Resultant powders and methods of making these powders may vary according to the following parameters, including, but not limited to, monolith thickness, initial drying time, heating temperature, duration of heating and duration of ball milling.

Hydrogel monoliths of varying thicknesses may be air dried then lyophilized. The lyophilized material may be ground using either a mortar and pestle or ball mill. The resulting powder is evaluated with and without a subsequent heating cycle at 50° C. for 45 minutes.

The newly formed hydrogel monoliths may be finely ground and then mixed with an equal volume of high molecular weight PEGs (oligomers or polymers of ethylene oxide, including, but not limited to, PEG3K or PEG5K) in the presence of a slight excess of buffer. The mixture may be vigorously stirred for several hours before drying and then being subjected to lyophilization. Coating the surface of hydrogel particles with large PEG molecules may enhance the dispersive properties of the resulting particles subsequent to lyophilization. Under some circumstances, PEG molecules irreversibly bind to the surface of TMOS derived hydrogels.

Tetramethoxysilane (TMOS) may be used as a foundation for hydrogel formation as described above. The following non-limiting combinations of components are contemplated:
TMOS+pVAX-hSlo;
TMOS+pVAX-hSlo+chitosan;
TMOS+pVAX-hSlo+PEG;
TMOS+pVAX-hSlo+PEG+chitosan;
TMOS contacted with monosubstituted organosilanes (e.g. alkyltrimethoxysilanes with the alkyl group being either methyl, ethyl or N-propyl)+pVAX-hSlo;
TMOS contacted with monosubstituted organosilanes (e.g. alkyltrimethoxysilanes with the alkyl group being either methyl, ethyl or N-propyl)+pVAX-hSlo+chitosan;
TMOS contacted with monosubstituted organosilanes (e.g. alkyltrimethoxysilanes with the alkyl group being either methyl, ethyl or N-propyl)+pVAX-hSlo+PEG;
TMOS contacted with monosubstituted organosilanes (e.g. alkyltrimethoxysilanes with the alkyl group being either methyl, ethyl or N-propyl)+pVAX-hSlo+glucose;
TMOS contacted with monosubstituted organosilanes (e.g. alkyltrimethoxysilanes with the alkyl group being either methyl, ethyl or N-propyl)+pVAX-hSlo+chitosan+PEG;
TMOS contacted with monosubstituted organosilanes (e.g. alkyltrimethoxysilanes with the alkyl group being either methyl, ethyl or N-propyl)+pVAX-hSlo+chitosan+glucose; and
TMOS contacted with monosubstituted organosilanes (e.g. alkyltrimethoxysilanes with the alkyl group being either methyl, ethyl or N-propyl)+pVAX-hSlo+PEG+glucose.

The strategy for this protocol is to tune the hydrophobicity of the interior of the particles by using small amounts of added alkylsubstituted silanes as a hydrophobic dopant in the sol-gel matrix (i.e. contacting an amount of alkylsubstituted silanes to a sol-gel matrix). This use of alkyl-substituted methoxysilanes generates sol-gels capable of enhancing the reactivity of encapsulated enzymes. These encapsulated enzymes have hydrophobic surfaces and may normally lose activity and stability in pure TMOS derived sol-gel matrices. Increasing the hydrophobicity of the interior of the particles may result in a slower release of pVAX-hSlo, thereby allowing for a sustained or more sustained delivery. Tuning the hydrophobicity of the particles may be desirable if non-aqueous delivery vehicles are used for the powders.

### Example 5

### In Vitro Characterization of Nanoparticles Containing

pVAX-hSlo plasmid is a nucleic acid with an absorbance peak at 260 nm. Therefore, release kinetics from the nanoparticles may be determined by change in absorbance. Freshly prepared nanoparticles containing the hSlo vectors are incubated in aqueous solution for varying amounts of time (e.g. between 0 and 24 hours). Subsequently, the nanoparticles are centrifuged and the release of nucleic acids into the supernatant is determined through absorbance. Quantitative-RT-PCR, using vector-specific primers, is performed for a further characterization of the release kinetics of the nucleic acid from the nanoparticle. Stability is tested by retaining nanoparticles containing the hSlo vectors for various periods of time (ranging from, for example, 1 day to three months (or 90 days)) and determining the release kinetics of the retained nanoparticles by the same method used for freshly prepared nanoparticles. Integrity of the released plasmids is determined by agarose gel electrophoresis followed by nucleic acid staining. The results of this analysis indicate the physical form of the nucleic acid released from the nanoparticles, e.g. circular, nicked or supercoiled. Furthermore, the released nucleic acid is subjected to restriction enzyme analysis.

### Example 6

### Topical Administration of Nanoparticle Delivery System

Nanoparticles of the disclosure were used to encapsulate the MaxiK for the present study. Data from this study demonstrate that the nanoparticles are capable of crossing the dermis. Rat models of ED showed demonstrable functional improvement following treatment.

Fluorescently-labeled nanoparticles were applied to the penis of rats under anesthesia. After one hour the rat was euthanized and the entire penis washed in phosphate buffered saline and fixed in 5% paraformaldehyde for 24 hours. Cross sections were taken at various points along the shaft of the penis. A typical result is shown in FIG. 8. Control animals (not treated with the nanoparticles) did not show any red spots. In all sections, spots could be observed at the dermis of the penis. The data indicate that these nanoparticles penetrated the dermis of the skin because washing and fixing of the penis would have removed external nanoparticles. Moreover, patches of red fluorescence could be seen in the corpora spongiosum and in the corpora vein.

Nanoparticles encapsulating erectogenic agents (NO or Sialorphin) facilitate erections in aging rats. The corpus cavernosum crus of nine month-old Sprague-Dawley rats was exposed and the intracorporal pressure (ICP) was measured using a 23-gauge needle inserted therein. After determining a steady baseline, a viscous solution of NO- or sialorphin-containing nanoparticles was applied to the shaft of the penis. Of note, the skin of the penis remained intact and at a different location to the site of measurement of ICP). Control animals were treated with “empty” nanoparticles, containing only phosphate buffer.

A total of 7 experimental animals were used in this initial study. In 5 of the 7 animals, there was a pronounced positive effect on the intracorporal pressure (ICP), resulting in a visible erection (tissue was prepared for histological analysis). Following histological analysis, there was no evidence of inflammation or congestion in these samples. Overall, the tissue appeared normal. These preliminary data demonstrate the ability of the engineered nanoparticles containing large molecules to cross “skin” barriers safely (without presentation of toxic effects).

### Example 7

### Biosafety/Biodistribution Profiles of Nanoparticles

There are two components to the nanoparticles: the nanoparticle and the hSlo vector. The biodistribution and pharmacokinetics of each of the components is determined. Pathology and histopathology analyses are performed to determine whether other organs are affected, and if so, which organs.

Pathology Determinations:

During the physiological studies to determine the effects of the nanoparticle encapsulated hSlo vectors on bladder function the animals will be monitored for potential systemic side-effects. Animals treated with the product and with nanoparticles encapsulating the empty vector (control) will be monitored for several physiological parameters related to vascular well-being, such as basal heart rate, systolic pressure, diastolic pressure and mean arterial pressure. A tail cuff system will be used, such as the CODA™2 mouse/rat tail cuff system from Kent Scientific Corp. (Torrington, Conn.) which allows non-invasive measurement of vascular physiological parameters. Following the physiological measurements animals will be euthanized and gross pathology will be performed. Sections of the bladder will be prepared for histology and examination. In particular, signs of vascular pathology or inflammation will be looked for.

Biodistribution: Nanoparticles containing the hSlo vector will be instilled in the bladder lumen of healthy anesthetized rats through the indwelling bladder catheter used for cystometry, as described in General Methods. Animals will then be euthanized at different time points (from 1 hour to 1 week) and tissues removed for determination of the presence/amount of the hSlo vector or nanoparticle. The main tissues to be investigated are the bladder, blood, heart, liver, kidney, brain, spleen, testis, lung, eye, prostate, axillary lymph node, epididymis, biceps, penis and colon. The amount (dose) of product administered to perform the biodistribution studies will be the same that has been shown in the studies of bladder function to induce the most significant physiological effect in reducing DO in PUO rats.

a) Nanoparticle detection: The nanoparticles used in the biodistribution experiments will be labelled either by conjugation with a fluorophore (FITC or DsRed) (as in FIG. 8) or biotinylated (to allow detection by antibodies). The organs cited above will be isolated and histological sections and tissue extracts will be prepared. For detection of biotinylated nanoparticles, immunohistochemistry and Western blot analysis of tissues will be performed using an antibody against the biotinylated nanoparticles, which would allow for quantification of nanoparticles in individual tissues by densitometric analysis of the images. For fluorescent nanoparticles, tissue sections will be examined by epifluorescence or confocal microscopy.

b) hSlo vector detection: Our laboratory has previously performed extensive biodistribution studies of pVAX-hSlo following its intracorporal injection in rats in order to satisfy the regulatory requirements of the Center for Biological Evaluation and Research (CBER) of the Federal Drug Administration (FDA). In these studies we used qRT-PCR to perform a temporal study of the plasmid distribution using primers for the kanamycin resistance gene of the pVAX vector. These studies were performed at various time points over the course of a week (4, 8, 24 hours and 1 week), which include the time points at which the physiological effect was determined. In the studies where the hSlo-nanoparticles were injected in the corpora, the plasmid could be detected in several tissues 4 hours after administration, though after one week its expression was restricted to the corpora. We expect that a similar time course study will be appropriate to determine the biodistribution of the hSlo vectors after intravesical administration. We will follow the same procedure to detect the hSlo vectors in the bladder tissue of PUO-treated rats. Bladders will be harvested after functional cystometric assessment, the urothelial and detrusor tissues will be separated under a dissecting microscope and tissues prepared for qRT-PCR analysis as previously described^{23,57}.

Monitoring Transfection Efficiency and hSlo Gene Expression in the Bladder:

Two components will determine the efficiency of transfection of cells targeted with the nanoparticles: uptake of the nanoparticles by cells and then expression of the encapsulated vector within transfected cells. Nanoparticle uptake will be monitored as describe above, using biotinylated or fluorescent-tagged nanoparticles, while cargo (vector) intracellular release will be determined by qRT-PCR targeting expression of the vectors' resistance genes. A similar approach, however, cannot be used to detect and monitor hSlo gene expression, given that it is already endogenously expressed in the bladder. To ascertain, therefore, that upon uptake of the product the cells are actually efficiently expressing the hSlo gene, we will tag the gene with the mCherry fluorescent reporter (red) and encapsulate the product with FITC-labelled nanoparticles (see FIG. 5B). This will allow us to simultaneously monitor the uptake and persistence of nanoparticles in the bladder (green fluorescence) and the hSlo expression (red fluorescence). The advantage of this approach is in that it will allow for both in vivo, ex vivo and in vitro monitoring (see preliminary data below, FIG. 9). Primers and antibodies for mCherry and FITC are also commercially available.

Preliminary Data:

In vitro studies performed with HeLa cells demonstrate that the efficiency of nanoparticle cellular uptake and expression of plasmids upon release from nanoparticles can be monitored using a fluorescent reporter gene. As show in FIG. 9A, shortly after addition of nanoparticles encapsulating a vector expressing mCherry, a high rate of transfection, approaching 95%, was observed in HeLa cells culture. We also demonstrate very high expression levels of the MaxiK gene in HEK293 transfected with nanoparticles encapsulating pMaxiK. HEK293 cells usually express very low levels of MaxiK (FIG. 9B). Even at the lowest amount of MaxiK-nanoparticle there is a 100,000-fold increase in gene expression after 20 h. The suitability of mCherry as a reporter for in vivo gene expression is shown in experiments in which we injected the bladder detrusor with pmCherry-N1. As shown in (FIG. 9C), mCherry fluorescence can be clearly detected in the pelvic region of the treated animal, and after removal of the bladders a heat map can be used to quantitate the expression (FIG. 9D).

Sample Size Considerations and Numbers of Animals:

For each biodistribution study 8 animals will be used for each of the five (5) time points. This number of animals is based on previous minimally acceptable numbers for biodistribution of pVAX-hSlo (accepted for safety studies for clinical trials by FDA) and also reasonable work level for analyzing 16 tissues from 8 animals in the second half period of the grant. A total of 40 female Sprague Dawley rats will be used in these experiments.

### Example 8

### Determination of Nanoparticles for Intravesical Delivery

The efficacy of intravesical therapy is potentially limited by the very low permeability of the urothelium and by drug dilution with urine and washout with micturition. Chemical and physical methods have been used to enhance drug absorption by temporarily disrupting the urothelial barrier. Use of these methods, however, can cause side-effects and tissue damage. Our goal in is to determine whether use of nanoparticles as a platform for intravesical delivery of the hSlo product will yield better therapeutic results than use of the hSlo alone to correct DO in PUO rats.

In this study, the plasmid construct that induced the most significant improvement in DO (and the nanoparticle with the best plasmid cargo loading capability, best tissue penetration and cargo release profile will be used to manufacture the new product in sufficent quantitities to be tested in the PUO model using the same methodology as decribed above. The nanoparticle preparation will be generated so that it contains the same quantity of naked vector to allow comparison between naked vector and the nanoparticle encapsulated vector.

The effects of the new product on bladder function of PUO rats will be evaluated based on cystometric parameters, as described above. Cystometric data will be compared to that obtained from animals treated with the naked vector. Statistical analysis will be performed decribed above. The experimental groups and number of animals to be used in this Example are shown in Table 3.

After cystometric evaluation the bladders from control treated and from nanoparticle+plasmid vector treated PUO rats will be harvested and used for ex vivo evaluation of changes in detrusor function by organ bath and path-clamping studies, as described above (see also General Methods).

TABLE 2
Number of animals per experimental group and doses for intravesical
treatment with control nanoparticles encapsulating the empty vector
and nanoparticles encapsulating the vector with the plasmid.

| | Dose (μg) | | |
|---|---|---|---|
| | 10 | 30 | 100 |
| Experimental Groups | Number of animals | | |
| Nanoparticle + empty vector (control) | 27 | 27 | 27 |
| Nanoparticle + plasmid vector | 27 | 27 | 27 |

### Other Embodiments

Although specific embodiments of the invention have been described herein for purposes of illustration, various modifications may be made without deviating from the spirit and scope of the disclosure. Accordingly, the invention is not limited except as by the appended claims.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### REFERENCES

1. Stewart W F, Van Rooyen J B, Cundiff G W, Abrams P, Herzog A R, Corey R, Hunt T L, Wein A J: Prevalence and burden of overactive bladder in the United States. World J. Urol. 2003; 20: 327-336
2. Abrams P, Cardozo L, Fall M, Griffiths D, Rosier P, Ulmsten U, van K P, Victor A, Wein A: The standardisation of terminology of lower urinary tract function: report from the Standardisation Sub-committee of the International Continence Society. Am. J. Obstet. Gynecol. 2002; 187: 116-126
3. Brading A F: The role of urinary urgency and its measurement in the overactive bladder symptom syndrome: current concepts and future prospects. BJU. Int. 2005; 96: 441-442
4. Andersson K E, Martin N, Nitti V: Selective beta-Adrenoceptor Agonists in the Treatment of Overactive Bladder. J. Urol. 2013
5. Angulo J C, Khullar V, Nitti V W, Siddiqui E: Evidence available on the use of the selective beta3-adrenoceptor agonist mirabegron for the treatment of overactive bladder. Actas Urol. Esp. 2013
6. Dmochowski R, Chapple C, Nitti V W, Chancellor M, Everaert K, Thompson C, Daniell G, Zhou J, Haag-Molkenteller C: Efficacy and safety of onabotulinumtoxinA for idiopathic overactive bladder: a double-blind, placebo controlled, randomized, dose ranging trial. J. Urol. 2010; 184: 2416-2422
7. Nitti V W, Dmochowski R, Herschorn S, Sand P, Thompson C, Nardo C, Yan X, Haag-Molkenteller C: OnabotulinumtoxinA for the treatment of patients with overactive bladder and urinary incontinence: results of a phase 3, randomized, placebo controlled trial. J. Urol. 2013; 189: 2186-2193
8. Nitti V W, Khullar V, van K P, Herschorn S, Cambronero J, Angulo J C, Blauwet M B, Dorrepaal C, Siddiqui E, Martin N E: Mirabegron for the treatment of overactive bladder: a prespecified pooled efficacy analysis and pooled safety analysis of three randomised, double-blind, placebo-controlled, phase III studies. Int. J. Clin. Pract. 2013; 67: 619-632
9. Nitti V W, Rosenberg S, Mitcheson D H, He W, Fakhoury A, Martin N E: Urodynamics and Safety of the beta-Adrenoceptor Agonist Mirabegron in Males with Lower Urinary Tract Symptoms and Bladder Outlet Obstruction. J. Urol. 2013
10. Meredith A L, Thorneloe K S, Werner M E, Nelson M T, Aldrich R W: Overactive bladder and incontinence in the absence of the B K large conductance Ca2+-activated K+ channel. J. Biol. Chem. 2004; 279: 36746-36752
11. Thorneloe K S, Meredith A L, Knorn A M, Aldrich R W, Nelson M T: Urodynamic properties and neurotransmitter dependence of urinary bladder contractility in the B K channel deletion model of overactive bladder. Am. J. Physiol Renal Physiol 2005; 289: F604-F610
12. Werner M E, Zvara P, Meredith A L, Aldrich R W, Nelson M T: Erectile dysfunction in mice lacking the large-conductance calcium-activated potassium (B K) channel. J. Physiol 2005; 567: 545-556
13. Melman A, Bar-Chama N, McCullough A, Davies K, Christ G: Plasmid-based gene transfer for treatment of erectile dysfunction and overactive bladder: results of a phase I trial. Isr. Med. Assoc. J. 2007; 9: 143-146
14. Babaoglu M, Zumrutbas A E, Acar I C, Hatip F B, Kucukatay V, Eskicorapci S, Aybek Z: Gap junction expression and the effects of gap junction inhibitors in overactive bladder models: does ovariectomy have a role? Int Urol. Nephrol. 2013
15. Imamura M, Negoro H, Kanematsu A, Yamamoto S, Kimura Y, Nagane K, Yamasaki T, Kanatani I, Ito N, Tabata Y, Ogawa O: Basic fibroblast growth factor causes urinary bladder overactivity through gap junction generation in the smooth muscle. Am. J. Physiol Renal Physiol 2009; 297: F46-F54
16. Miyazato M, Sugaya K, Nishijima S, Kadekawa K, Machida N, Oshiro Y, Saito S: Changes of bladder activity and connexin 43-derived gap junctions after partial bladder-outlet obstruction in rats. Int Urol. Nephrol. 2009; 41: 815-821
17. Negoro H, Kanematsu A, Imamura M, Kimura Y, Matsuoka R, Tanaka M, Tabata Y, Ogawa O: Regulation of connexin 43 by basic fibroblast growth factor in the bladder: transcriptional and behavioral implications. J. Urol. 2011; 185: 2398-2404
18. Suadicani S O, Urban-Maldonado M, Tar M T, Melman A, Spray D C: Effects of ageing and streptozotocin-induced diabetes on connexin43 and P2 purinoceptor expression in the rat corpora cavernosa and urinary bladder. BJU Int 2009; 103: 1686-93
19. Wang H, Lee S W, Day N S, Christ G J: Gap junction channel activity in cultured human bladder smooth muscle cell pairs: gating and unitary conductances. Urology 2001; 57: 111
20. Christ G J, Hodges S: Molecular mechanisms of detrusor and corporal myocyte contraction: identifying targets for pharmacotherapy of bladder and erectile dysfunction. Br J Pharmacol 2006; 147 Suppl 2: S41-55
21. Li M, Sun Y, Simard J M, Wang J Y, Chai T C: Augmented bladder urothelial polyamine signaling and block of B K channel in the pathophysiology of overactive bladder syndrome. American Journal of Physiology—Cell Physiology 2009; 297: C1445-C1451
22. Wang Y, Tar M T, Fu S, Melman A, Davies K P: Diabetes attenuates urothelial modulation of detrusor contractility and spontaneous activity. Int J Urol 2014: Available On-line ahead of print.
23. Christ G J, Andersson K E, Williams K, Zhao W, D'Agostino R, Jr., Kaplan J, Aboushwareb T, Yoo J, Calenda G, Davies K P, Sellers R S, Melman A: Smooth-muscle-specific gene transfer with the human maxi-k channel improves erectile function and enhances sexual behavior in atherosclerotic cynomolgus monkeys. Eur Urol 2009; 56: 1055-66
24. Gordon E, Semus S F, Lozinskaya I M, Lin Z, Xu X: Characterizing the role of Thr352 in the inhibition of the large conductance Ca2+-activated K+ channels by 1-[1-hexyl-6-(methyloxy)-1H-indazol-3-yl]-2-methyl-1-propanone. J. Pharmacol. Exp. Ther. 2010; 334: 402-409
25. Han G, Tar M, Kuppam D S, Friedman A, Melman A, Friedman J, Davies K P: Nanoparticles as a novel delivery vehicle for therapeutics targeting erectile dysfunction. J Sex Med 2010; 7: 224-33
26. Friedman A J, Han G, Navati M S, Chacko M, Gunther L, Alfieri A, Friedman J M: Sustained release nitric oxide releasing nanoparticles: characterization of a novel delivery platform based on nitrite containing hydrogel/glass composites. Nitric. Oxide. 2008; 19: 12-20
27. Han G, Martinez L R, Mihu M R, Friedman A J, Friedman J M, Nosanchuk J D: Nitric oxide releasing nanoparticles are therapeutic for Staphylococcus aureus abscesses in a murine model of infection. PLoS. One. 2009; 4: e7804
28. Schairer D, Martinez L R, Blecher K, Chouake J, Nacharaju P, Gialanella P, Friedman J M, Nosanchuk J D, Friedman A: Nitric oxide nanoparticles: pre-clinical utility as a therapeutic for intramuscular abscesses. Virulence. 2012; 3: 62-67
29. Sanchez D A, Schairer D, Tuckman-Vernon C, Chouake J, Kutner A, Makdisi J, Friedman J M, Nosanchuk J D, Friedman A J: Amphotericin B releasing nanoparticle topical treatment of Candida spp. in the setting of a burn wound. Nanomedicine. 2013
30. Cabrales P, Han G, Roche C, Nacharaju P, Friedman A J, Friedman J M: Sustained release nitric oxide from long-lived circulating nanoparticles. Free Radic. Biol. Med. 2010; 49: 530-538
31. Nachuraju P, Friedman A J, Friedman J M, Cabrales P: Exogenous nitric oxide prevents cardiovascular collapse during hemorrhagic shock. Resuscitation 2011; 82: 607-613
32. Nacharaju P, Tuckman-Vernon C, Maier K E, Chouake J, Friedman A, Cabrales P, Friedman J M: A nanoparticle delivery vehicle for S-nitroso-N-acetyl cysteine: sustained vascular response. Nitric. Oxide. 2012; 27: 150-160
33. Cabrales P, Han G, Nacharaju P, Friedman A J, Friedman J M: Reversal of hemoglobin-induced vasoconstriction with sustained release of nitric oxide. Am J Physiol Heart Circ Physiol 2011; 300: H49-56
34. Hodges S J, Zhou G, Deng F M, Aboushwareb T, Turner C, Andersson K E, Santago P, Case D, Sun T T, Christ G J: Voiding pattern analysis as a surrogate for cystometric evaluation in uroplakin II knockout mice. J Urol 2008; 179: 2046-51
35. Andersson K E: Animal in vivo models of lower urinary tract dysfunction: translational value. J. Urol. 2013; 189: 1622-1623
36. Chang S, Gomes C M, Hypolite J A, Marx J, Alanzi J, Zderic S A, Malkowicz B, Wein A J, Chacko S: Detrusor overactivity is associated with downregulation of large-conductance calcium- and voltage-activated potassium channel protein. Am. J. Physiol Renal Physiol 2010; 298: F1416-F1423
37. Christ G J H Y, Zhao W, Schenk G, Venkateswarlu K, Wang H Z, Tar M T, Melman A.: Effects of streptozotocin-induced diabetes on bladder and erectile (dys)function in the same rat in vivo, BJU, 2006, pp 1076-1083
38. Jin L H, Andersson K E, Han J U, Kwon Y H, Park C S, Shin H Y, Yoon S M, Lee T: Persistent detrusor overactivity in rats after relief of partial urethral obstruction. Am. J. Physiol Regul. Integr. Comp Physiol 2011; 301: R896-R904
39. Melman A, Tar M, Boczko J, Christ G, Leung A C, Zhao W, Russell R G: Evaluation of two techniques of partial urethral obstruction in the male rat model of bladder outlet obstruction. Urology 2005; 66: 1127-1133
40. Melman A, Zotova E, Kim M, Arezzo J, Davies K, DiSanto M, Tar M: Longitudinal studies of time-dependent changes in both bladder and erectile function after streptozotocin-induced diabetes in Fischer 344 male rats. BJU. Int. 2009; 104: 1292-1300
41. Davies K P, Zhao W, Tar M, Figueroa J C, Desai P, Verselis V K, Kronengold J, Wang H Z, Melman A, Christ G J: Diabetes-induced changes in the alternative splicing of the slo gene in corporal tissue. Eur. Urol. 2007; 52: 1229-1237
42. Wang H Z, Day N, Valcic M, Hsieh K, Serels S, Brink P R, Christ G J: Intercellular communication in cultured human vascular smooth muscle cells. Am J Physiol Cell Physiol 2001; 281: C75-88
43. Wang H Z, Lee S W, Christ G J: Comparative studies of the maxi-K (K(Ca)) channel in freshly isolated myocytes of human and rat corpora. Int J Impot Res 2000; 12: 9-18
44. Gordon E, S emus S F, Lozinskaya I M, Lin Z, Xu X: Characterizing the role of Thr352 in the inhibition of the large conductance Ca2+-activated K+ channels by 1-[1-hexyl-6-(methyloxy)-1H-indazol-3-yl]-2-methyl-1-propanone. J Pharmacol Exp Ther 2010; 334: 402-9
45. Kinder R B, Mundy A R: Pathophysiology of idiopathic detrusor instability and detrusor hyper-reflexia. An in vitro study of human detrusor muscle. Br J Urol 1987; 60: 509-15
46. Mills I W, Greenland J E, McMurray G, McCoy R, Ho K M, Noble J G, Brading A F: Studies of the pathophysiology of idiopathic detrusor instability: the physiological properties of the detrusor smooth muscle and its pattern of innervation. J Urol 2000; 163: 646-51
47. Banks F C, Knight G E, Calvert R C, Morgan R J, Burnstock G: Alterations in purinergic and cholinergic components of contractile responses of isolated detrusor contraction in a rat model of partial bladder outlet obstruction. BJU Int 2006; 97: 372-8
48. Milicic I, Buckner S A, Daza A, Coghlan M, Fey T A, Brune M E, Gopalakrishnan M: Pharmacological characterization of urinary bladder smooth muscle contractility following partial bladder outlet obstruction in pigs. Eur J Pharmacol 2006; 532: 107-14
49. Oger S, Behr-Roussel D, Gorny D, Bernabe J, Comperat E, Chartier-Kastler E, Denys P, Giuliano F: Effects of potassium channel modulators on myogenic spontaneous phasic contractile activity in human detrusor from neurogenic patients. BJU Int 2011; 108: 604-11
50. Petkov G V: Role of potassium ion channels in detrusor smooth muscle function and dysfunction. Nat Rev Urol 2012; 9: 30-40
51. Karicheti V, Christ G J: Physiological roles for K+ channels and gap junctions in urogenital smooth muscle: implications for improved understanding of urogenital function, disease and therapy. Curr Drug Targets 2001; 2: 1-20
52. Hypolite J A, Lei Q, Chang S, Zderic S A, Butler S, Wein A J, Malykhina A P, Chacko S: Spontaneous and evoked contractions are regulated by PKC-mediated signaling in detrusor smooth muscle: involvement of B K channels. Am J Physiol Renal Physiol 2013; 304: F451-62
53. Vahabi B, McKay N G, Lawson K, Sellers D J: The role of c-kit-positive interstitial cells in mediating phasic contractions of bladder strips from streptozotocin-induced diabetic rats. BJU Int 2011; 107: 1480-7
54. Stevens L A, Sellers D J, McKay N G, Chapple C R, Chess-Williams R: Muscarinic receptor function, density and G-protein coupling in the overactive diabetic rat bladder. Auton Autacoid Pharmacol 2006; 26: 303-9
55. Tammela T L, Briscoe J A, Levin R M, Longhurst P A: Factors underlying the increased sensitivity to field stimulation of urinary bladder strips from streptozotocin-induced diabetic rats. Br J Pharmacol 1994; 113: 195-203
56. Liong C, Ortiz D, Ao-ieong E, Navati M S, Friedman J M, Cabrales P: Localized increase of tissue oxygen tension by magnetic targeted drug delivery. Nanotechnology 2014; 25: 265102
57. Melman A, Zhao W, Davies K P, Bakal R, Christ G J: The successful long-term treatment of age related erectile dysfunction with hSlo cDNA in rats in vivo. J. Urol. 2003; 170: 285-290

1. A nucleic acid molecule comprising the nucleic acid sequence of SEQ ID NO:3 wherein the nucleic acid has a single point mutation at nucleotide position 1054 wherein said point mutation results in serine at position 352 of SEQ ID No: 4.

2. The nucleic acid molecule of claim 1 operably-linked to a promoter.

3. The nucleic acid molecule of claim 2, wherein the promoter is not an urothelium specific expression promoter.

4. The nucleic acid molecule of claim 2, wherein the promoter is a CMV promoter or a smooth muscle specific expression promoter.

5. A plasmid comprising the nucleic acid molecule of claim 1.

6. The nucleic acid molecule of claim 1, or the plasmid of claim 5 wherein said nucleic acid molecule or plasmid is associated with or conjugated to a nanoparticle.

7. A nanoparticle comprising the plasmid of claim 5.

8. A delivery system comprising a plurality of nanoparticles of claim 7, and a pharmaceutically acceptable diluent or carrier.

9. A vector comprising the nucleic acid molecule of claim 1.

10. The vector of claim 9, wherein said vector is an adenovirus.

11. A delivery system comprising a plurality of vectors of claim 9, and a pharmaceutically acceptable diluent or carrier.

12. The delivery system of claim 8 or 11, wherein the delivery system is suitable for topical administration to a subject.

13. The delivery system of claim 8 or 11, wherein the delivery system is suitable for systemic administration to a subject.

14. A method for expressing a variant BKα channel within a smooth muscle cell, comprising contacting the cell the nucleic acid molecule of claim 1.

15. The method of claim 14, wherein the cell is contacted in vivo, ex vivo, or in vitro.

16. The method of claim 14, wherein the smooth muscle is a detrusor urinae muscle.

17. A method of treating smooth muscle dysfunction in a subject, comprising introducing into smooth muscle cells of the subject the nucleic acid molecule of claim 1 or the delivery system of claim 8 or 11, wherein the nucleic acid is expressed in the smooth cells such that smooth muscle tone is regulated, and wherein the regulation of smooth muscle tone results in less heightened contractility of smooth muscle in the subject.

18. The method of claim 17, wherein the subject has over active bladder (OAB) syndrome, erectile dysfunction (ED), asthma; benign hyperplasia of the prostate gland (BHP); coronary artery disease (infused during angiography); genitourinary dysfunctions of the bladder, endopelvic fascia, prostate gland, ureter, urethra, urinary tract, and vas deferens; irritable bowel syndrome; migraine headaches; premature labor; Raynaud's syndrome; and thromboangitis obliterans.

19. The method of claim 17, wherein the nucleic acid molecule is introduced by naked DNA transfer.

20. The method of claim 17 wherein the delivery system is introduced by instillation into the lumen of the bladder.

21. A method of treating over active bladder (OAB) syndrome in a subject, comprising introducing into bladder smooth muscle cells of the subject the nucleic acid molecule of claim 1 or the delivery system of claim 8 or 11, wherein the nucleic acid is expressed in the bladder smooth cells such that bladder smooth muscle tone is regulated, and wherein the regulation of bladder smooth muscle tone results in less heightened contractility of smooth muscle in the subject.

22. A method for treating penile flaccidity caused by heightened contractility of penile smooth muscle in a subject, comprising introducing into penile smooth muscle cells of the subject a the nucleic acid molecule of claim 1 or the delivery system of claim 8 or 11, wherein the nucleic acid expressed in the penile smooth muscle cells such that penile smooth muscle tone is regulated, and wherein the regulation of penile smooth muscle tone results in less heightened contractility of penile smooth muscle in the subject.

23. The method of claim 21, wherein the nucleic acid molecule is introduced by naked DNA transfer.

24. The method of claim 21, wherein the delivery system is introduced by instillation into the lumen of the bladder.
